SEQUENCE LISTING (33 sequences)

SEQ ID NO: 1 - DNA, 20 bp, Artificial sequence
  Single strand DNA oligonucleotide
atggtattggaagctacagt

SEQ ID NO: 2 - DNA, 28 bp, Artificial sequence
  Single strand DNA oligonucleotide
ttgtcgactcaacctctcaatctcaaga

SEQ ID NO: 3 - DNA, 42 bp, Artificial sequence
  Single strand DNA oligonucleotide
ggaaaggttaagacagcagcaaggtggaggcaagactttgac

SEQ ID NO: 4 - DNA, 20 bp, Artificial sequence
  Single strand DNA oligonucleotide
tcacaagtataaaccttcta

SEQ ID NO: 5 - DNA, 40 bp, Artificial sequence
  Single strand DNA oligonucleotide
tgtttatccaacccaccgatgcggtaaacgtagataacac

SEQ ID NO: 6 - DNA, 40 bp, Artificial sequence
  Single strand DNA oligonucleotide
tttttgaagctggcagcaccatcggtggcttagaatctca

SEQ ID NO: 7 - DNA, 40 bp, Artificial sequence
  Single strand DNA oligonucleotide
tgagattctaagccaccgatggtgctgccagcttcaaaaa

SEQ ID NO: 8 - DNA, 43 bp, Artificial sequence
  Single strand DNA oligonucleotide
ggaaaggttaagacagcagcaatggtggaggcaagactttgac

SEQ ID NO: 9 - DNA, 44 bp, Artificial sequence
  Single strand DNA oligonucleotide
ggaaaggttaagacagcagcaattggtggaggcaagactttgac

SEQ ID NO: 10 - DNA, 45 bp, Artificial sequence
  Single strand DNA oligonucleotide
ggaaaggttaagacagcagcaatttggtggaggcaagactttgac

SEQ ID NO: 11 - DNA, 40 bp, Artificial sequence
  Single strand DNA oligonucleotide
gtgttatctacgtttaccgcatcggtgggttggataaaca

SEQ ID NO: 12 - DNA, 20 bp, Artificial sequence
  Single strand DNA oligonucleotide
attagttccgtcacaggggc

SEQ ID NO: 13 - DNA, 20 bp, Artificial sequence
  Single strand DNA oligonucleotide
gttcgggtggaagatcttgg

SEQ ID NO: 14 - DNA, 40 bp, Artificial sequence
  Single strand DNA oligonucleotide
gaaagatcaatgggcagctgagctgggcatccgacacgta

SEQ ID NO: 15 - DNA, 18 bp, Artificial sequence
  Single strand DNA oligonucleotide
tacgagccggaagcataa

SEQ ID NO: 16 - DNA, 21 bp, Artificial sequence
  Single strand DNA oligonucleotide
ttgctgctgtcttaacctttc

SEQ ID NO: 17 - DNA, 37 bp, Artificial sequence
  Single strand DNA oligonucleotide
gctgcaaggcgattaagtatggtattggaagctacag

SEQ ID NO: 18 - DNA, 37 bp, Artificial sequence
  Single strand DNA oligonucleotide
ctgtagcttccaataccatacttaatcgccttgcagc

SEQ ID NO: 19 - DNA, 20 bp, Artificial sequence
  Single strand DNA oligonucleotide
ggtgctgccagcttcaaaaa

SEQ ID NO: 20 - DNA, 238 bp, Artificial sequence
  Ubiquitin
aaaagcttggtggaggcaagactttgactggtaagaccatcactttggaagttgaatcttctgacactattgacaatgtcaagtcaaagattcaagacaaggaaggtatcccacctgaccaacaaagattgatctttgctggtaagcaattggaagacggtagaaccttgtctgactacaacattcaaaaagaatccactttgcacttagtcttgagattgagaggttgagtcgacaa

SEQ ID NO: 21 - DNA, 750 bp, Artificial sequence
  Saccharomyces cerevisiae derived RPN10 gene sequence
atggtattggaagctacagtgttagtgattgataattcagagtactctcgcaatggcgactttcctaggacaaggtttgaagcccagatcgactcggtagagttcatatttcaagccaagagaaacagcaatcctgagaatacagtgggtcttatctctggtgctggcgccaaccccagggtgttatctacgtttaccgccgagtttgggaagattcttgctggactacacgacacgcagatcgagggtaagctgcatatggccactgcgttgcagatcgctcagctgactttgaagcatcgccagaataaggtccaacatcaaaggattgtggcatttgtgtgtagcccaataagtgattctcgagacgaattgatcagattggcaaaaacactgaaaaagaataatgttgccgtggacatcatcaattttggagagattgaacagaacacggagcttttggatgagttcatagctgcagtgaacaaccctcaagaagaaactagtcatttgcttactgtgacgcctggccccagactgctgtacgagaacatcgcatcttcacccataattctcgaagaaggatcctccggtatgggcgcctttggtgggtctggcggtgattccgatgccaatggcacatttatggacttcggggtagacccatcaatggacccagaactggcaatggccttgcgtctgtctatggaagaagagcagcaaagacaggaaaggttaagacagcagcaa

SEQ ID NO: 22 - DNA, 980 bp, Artificial sequence
  RPN10-ubiquitin fusion
atggtattggaagctacagtgttagtgattgataattcagagtactctcgcaatggcgactttcctaggacaaggtttgaagcccagatcgactcggtagagttcatatttcaagccaagagaaacagcaatcctgagaatacagtgggtcttatctctggtgctggcgccaaccccagggtgttatctacgtttaccgccgagtttgggaagattcttgctggactacacgacacgcagatcgagggtaagctgcatatggccactgcgttgcagatcgctcagctgactttgaagcatcgccagaataaggtccaacatcaaaggattgtggcatttgtgtgtagcccaataagtgattctcgagacgaattgatcagattggcaaaaacactgaaaaagaataatgttgccgtggacatcatcaattttggagagattgaacagaacacggagcttttggatgagttcatagctgcagtgaacaaccctcaagaagaaactagtcatttgcttactgtgacgcctggccccagactgctgtacgagaacatcgcatcttcacccataattctcgaagaaggatcctccggtatgggcgcctttggtgggtctggcggtgattccgatgccaatggcacatttatggacttcggggtagacccatcaatggacccagaactggcaatggccttgcgtctgtctatggaagaagagcagcaaagacaggaaaggttaagacagcagcaaggtggaggcaagactttgactggtaagaccatcactttggaagttgaatcttctgacactattgacaatgtcaagtcaaagattcaagacaaggaaggtatcccacctgaccaacaaagattgatctttgctggtaagcaattggaagacggtagaaccttgtctgactacaacattcaaaaagaatccactttgcacttagtcttgagattgagaggttgagtcgacaa

SEQ ID NO: 23 - DNA, 200 bp, Artificial sequence
  S. cerevisiae derived RPN10(IV)  proteasomal subunit
atggtattggaagctacagtgttagtgattgataattcagagtactctcgcaatggcgactttcctaggacaaggtttgaagcccagatcgactcggtagagttcatatttcaagccaagagaaacagcaatcctgagaatacagtgggtcttatctctggtgctggcgccaaccccagggtgttatctacgtttaccgc

SEQ ID NO: 24 - DNA, 200 bp, Artificial sequence
  S. cerevisiae derived RPT5 proteasomal subunit
atcggtgggttggataaacagattgaagagctggtggaggccattgtgttacctatgaaacgtgcagataaattcaaagatatgggaattagagcgcccaagggtgccctaatgtacgggcctccgggtacaggtaaaactttattagctcgtgcttgcgcggctcagacgaatgctacttttttgaagctggcagcacc

SEQ ID NO: 25 - DNA, 765 bp, Artificial sequence
  S. cerevisiae derived RPT2 proteasomal subunit
atcggtggcttagaatctcaaattcaagaaataaaagagtctgtagagttaccgttaacacaccccgaactgtatgaagagatgggtatcaaacctcccaagggtgtgattctttatggtgcacccggtacaggtaagacattgctagcaaaggctgttgcaaatcaaacatctgccacattcttaaggattgttggctcagagttaattcaaaaatatttgggggatggtcctaggttgtgtaggcaaattttcaaagtagcgggtgagaatgcaccaagtattgtatttattgatgaaatagacgccattggtactaaaagatatgactccaatagtggtggtgagagagaaattcagagaaccatgctggaattgctgaaccagctggacgggttcgacgacagaggggatgtgaaggtgattatggccactaacaaaatcgagactcttgacccagcattaatcaggcctgggagaattgatcgtaaaatcttatttgaaaaccctgatctaagtactaagaaaaaaattttagggattcacacttccaagatgaatttaagtgaggacgttaatcttgaaactttagtaacaacaaaggatgatttgtcaggcgccgatatccaggcaatgtgtacagaagcaggtttgttagcccttagagagagaagaatgcaagttacagcggaggattttaaacaagccaaggagagagtcatgaaaaataaagttgaggaaaatttagaaggtttatacttgtga

SEQ ID NO: 26 - DNA, 1165 bp, Artificial sequence
  RPT10-RPT5-RPT2 fusion sequence
atggtattggaagctacagtgttagtgattgataattcagagtactctcgcaatggcgactttcctaggacaaggtttgaagcccagatcgactcggtagagttcatatttcaagccaagagaaacagcaatcctgagaatacagtgggtcttatctctggtgctggcgccaaccccagggtgttatctacgtttaccgcatcggtgggttggataaacagattgaagagctggtggaggccattgtgttacctatgaaacgtgcagataaattcaaagatatgggaattagagcgcccaagggtgccctaatgtacgggcctccgggtacaggtaaaactttattagctcgtgcttgcgcggctcagacgaatgctacttttttgaagctggcagcaccatcggtggcttagaatctcaaattcaagaaataaaagagtctgtagagttaccgttaacacaccccgaactgtatgaagagatgggtatcaaacctcccaagggtgtgattctttatggtgcacccggtacaggtaagacattgctagcaaaggctgttgcaaatcaaacatctgccacattcttaaggattgttggctcagagttaattcaaaaatatttgggggatggtcctaggttgtgtaggcaaattttcaaagtagcgggtgagaatgcaccaagtattgtatttattgatgaaatagacgccattggtactaaaagatatgactccaatagtggtggtgagagagaaattcagagaaccatgctggaattgctgaaccagctggacgggttcgacgacagaggggatgtgaaggtgattatggccactaacaaaatcgagactcttgacccagcattaatcaggcctgggagaattgatcgtaaaatcttatttgaaaaccctgatctaagtactaagaaaaaaattttagggattcacacttccaagatgaatttaagtgaggacgttaatcttgaaactttagtaacaacaaaggatgatttgtcaggcgccgatatccaggcaatgtgtacagaagcaggtttgttagcccttagagagagaagaatgcaagttacagcggaggattttaaacaagccaaggagagagtcatgaaaaataaagttgaggaaaatttagaaggtttatacttgtga

SEQ ID NO: 27 - DNA, 58 bp, Artificial sequence
  Active Site Loop (ASL) of E2-25K
attagttccgtcacaggggctatttgtttggatatcctgaaagatcaatgggcagctg

SEQ ID NO: 28 - DNA, 6036 bp, Artificial sequence
  E2-S ubiquitin ligase cloned into pET-22 plasmid
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcataactccaacgtggagaacctacccccgcacatcatccgcctggtgtacaaggaggtgacgacactgaccgcagacccacccgatggcatcaaggtctttcccaacgaggaggacctcaccgacctccaggtcaccatcgagggccctgaggggaccccatatgctggaggtctgttccgcatgaaactcctgctggggaaggacttccctgcctccccacccaagggctacttcctgaccaagatcttccacccgaacgtgggcgccaatggcgagatctgcgtcaacgtgctcaagagggactggacggctgagctgggcatccgacacgtactgctgaccatcaagtgcctgctgatccaccctaaccccgagtctgcactcaacgaggaggcgggccgcctgctcttggagaactacgaggagtatgcggctcgggcccgtctgctcacagagatccacgggggcgccggcgggcccagcggcagggccgaagccggtcgggccctggccagtggcactgaagcttcctccaccgaccctggggccccagggggcccgggaggggctgagggtcccatggccaagaagcatgctggcgagcgcgataagaagctggcggccaagaaaaagacggacaagaagcgggcgctgcggcggctgctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 29 - DNA, 6039 bp, Artificial sequence
  E2-S-25K-ASL fusion cloned in pET-22 plasmid
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcataactccaacgtggagaacctacccccgcacatcatccgcctggtgtacaaggaggtgacgacactgaccgcagacccacccgatggcatcaaggtctttcccaacgaggaggacctcaccgacctccaggtcaccatcgagggccctgaggggaccccatatgctggaggtctgttccgcatgaaactcctgctggggaaggacttccctgcctccccacccaagggctacttcctgaccaagatcttccacccgaacattagttccgtcacaggggctatttgtttggatatcctgaaagatcaatgggcagctgagctgggcatccgacacgtactgctgaccatcaagtgcctgctgatccaccctaaccccgagtctgcactcaacgaggaggcgggccgcctgctcttggagaactacgaggagtatgcggctcgggcccgtctgctcacagagatccacgggggcgccggcgggcccagcggcagggccgaagccggtcgggccctggccagtggcactgaagcttcctccaccgaccctggggccccagggggcccgggaggggctgagggtcccatggccaagaagcatgctggcgagcgcgataagaagctggcggccaagaaaaagacggacaagaagcgggcgctgcggcggctgctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 30 - DNA, 2964 bp, Artificial sequence
  Bluescript-KS plasmid sequence
ggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 31 - DNA, 3418 bp, Artificial sequence
  RPN10 sequence cloned into the KS plasmid
tacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagtatggtattggaagctacagtgttagtgattgataattcagagtactctcgcaatggcgactttcctaggacaaggtttgaagcccagatcgactcggtagagttcatatttcaagccaagagaaacagcaatcctgagaatacagtgggtcttatctctggtgctggcgccaaccccagggtgttatctacgtttaccgccgagtttgggaagattcttgctggactacacgacacgcagatcgagggtaagctgcatatggccactgcgttgcagatcgctcagctgactttgaagcatcgccagaataaggtccaacatcaaaggattgtggcatttgtgtgtagcccaataagtgattctcgagacgaattgatcagattggcaaaaacactgaaaaagaataatgttgccgtggacatcatcaattttggagagattgaacagaacacggagcttttggatgagttcatagctgcagtgaacaaccctcaagaagaaactagtcatttgcttactgtgacgcctggccccagactgctgtacgagaacatcgcatcttcacccataattctcgaagaaggatcctccggtatgggcgcctttggtgggtctggcggtgattccgatgccaatggcacatttatggacttcggggtagacccatcaatggacccagaactggcaatggccttgcgtctgtctatggaagaagagcagcaaagacaggaaaggttaagacagcagcaa

SEQ ID NO: 32 - DNA, 400 bp, Artificial sequence
  RPN10-RPT5 fusion construct
atggtattggaagctacagtgttagtgattgataattcagagtactctcgcaatggcgactttcctaggacaaggtttgaagcccagatcgactcggtagagttcatatttcaagccaagagaaacagcaatcctgagaatacagtgggtcttatctctggtgctggcgccaaccccagggtgttatctacgtttaccgcatcggtgggttggataaacagattgaagagctggtggaggccattgtgttacctatgaaacgtgcagataaattcaaagatatgggaattagagcgcccaagggtgccctaatgtacgggcctccgggtacaggtaaaactttattagctcgtgcttgcgcggctcagacgaatgctacttttttgaagctggcagcacc

SEQ ID NO: 33 - DNA, 981 bp, Artificial sequence
  Rpn10-t-ubiquitin fusion construct
atggtattggaagctacagtgttagtgattgataattcagagtactctcgcaatggcgactttcctaggacaaggtttgaagcccagatcgactcggtagagttcatatttcaagccaagagaaacagcaatcctgagaatacagtgggtcttatctctggtgctggcgccaaccccagggtgttatctacgtttaccgccgagtttgggaagattcttgctggactacacgacacgcagatcgagggtaagctgcatatggccactgcgttgcagatcgctcagctgactttgaagcatcgccagaataaggtccaacatcaaaggattgtggcatttgtgtgtagcccaataagtgattctcgagacgaattgatcagattggcaaaaacactgaaaaagaataatgttgccgtggacatcatcaattttggagagattgaacagaacacggagcttttggatgagttcatagctgcagtgaacaaccctcaagaagaaactagtcatttgcttactgtgacgcctggccccagactgctgtacgagaacatcgcatcttcacccataattctcgaagaaggatcctccggtatgggcgcctttggtgggtctggcggtgattccgatgccaatggcacatttatggacttcggggtagacccatcaatggacccagaactggcaatggccttgcgtctgtctatggaagaagagcagcaaagacaggaaaggttaagacagcagcaatggtggaggcaagactttgactggtaagaccatcactttggaagttgaatcttctgacactattgacaatgtcaagtcaaagattcaagacaaggaaggtatcccacctgaccaacaaagattgatctttgctggtaagcaattggaagacggtagaaccttgtctgactacaacattcaaaaagaatccactttgcacttagtcttgagattgagaggttgagtcgacaa

(19) United States
(12) Patent Application Publication
(10) Pub. No.: US 2013/0316358 A1
(43) Pub. Date: Nov. 28, 2013

(54) METHODS OF DIAGNOSING DISEASE USING OVERLAP EXTENSION PCR

(71) Applicants: Ami Navon, Yavne (IL); Lior Zelcbuch, Rehovot (IL)

(72) Inventors: Ami Navon, Yavne (IL); Lior Zelcbuch, Rehovot (IL)

(73) Assignee: YEDA RESEARCH AND DEVELOPMENT CO. LTD., Rehovot (IL)

(21) Appl. No.: 13/982,809

(22) PCT Filed: Jan. 31, 2012
(86) PCT No.: PCT/IL12/50033

§ 371 Date: Jul. 31, 2013

(51) Int. Cl. C12Q 1/68

(52) U.S. Cl. CPC C12Q 1/6853
USPC 435/6.12; 435/91.2; 536/24.33

(57) ABSTRACT

A method of diagnosing a disease in a subject in need thereof is disclosed. The method comprises:
(a) linking a plurality of non-contiguous DNA fragments from a sample of the subject to generate a polynucleotide product, the linking being effected by contacting the plurality of non-contiguous DNA fragments with a multiplex overlap-extension primer mix under conditions that allow simultaneous linkage of the DNA fragments and amplification of the polynucleotide product, wherein the primer mix comprises two flanking primers and at least one linker primer; and
(b) determining a sequence of the polynucleotide product, wherein the sequence is indicative of the disease.

### FIELD AND BACKGROUND OF THE INVENTION

The present invention, in some embodiments thereof, relates to methods of diagnosing a disease or susceptibility thereto based on DNA sequencing.

Mutations/variations in the human genome are involved in most diseases, going from monogenetic to multifactorial diseases, and acquired diseases such as cancer. Even the susceptibility to infectious diseases, and the response to pharmaceutical drugs, is affected by the composition of an individual's genome. Most genetic tests, which screen for such mutations/variations, require amplification of the DNA region under investigation. However, the size of the genomic DNA that can be amplified is rather limited. For example, the upper size limit of an amplified DNA fragment in a standard PCR reaction is about 2 kb. This contrasts sharply with the total size of 3 billion nucleotides of which the human genome is build up. As more and more mutations/variations are found to be involved in disease, there is a need for robust assays in which different DNA regions, that harbor the different mutations/variations, are analyzed together.

Thus, in some cases it is necessary or at least desirable to perform a number of tests, for example, evaluating the sequence of a large number of mutations or single nucleotide polymorphisms (SNPs) in an individual's gDNA. For example, the cystic fibrosis or CFTR gene (approximately 5 kb long), contains approximately 1,300 rare mutations and polymorphisms and it may be desirable to determine the nucleotide sequence at many if not all of the potential mutation and/or SNP sites in a particular individual's gDNA.

In a classical multiplex PCR reaction, different fragments are amplified in a single tube, simply by adding all pairs of amplicon-specific primers to a reaction mixture. The higher the number of primers that are combined in a single PCR reaction, the higher the chance that particular primer interactions (such as primer-dimerization), and a specific primer/template interactions occur, so that particular amplicons fail to amplify. There is thus a limitation in the number of amplicons that can be co-amplified when primers are simply mixed with long stretches of DNA template.

U.S. Pat. No. 7,749,697 teaches multiplex overlap-extension RT-PCR to link two or more nucleotide sequences encoding for domains or subunits of a heteromeric protein in a single reaction. The method especially relates to the linkage of variable regions encoding sequences from, for example, immunoglobulins, T cell receptors, or B cell receptors.

Additional background art includes: Heckman, K. L. & Pease, L. R. Nat Protoc 2, 924-932; Ho, S. N., Hunt, H. D., Horton, R. M., Pullen, J. K. & Pease, L. R. Gene 77, 51-59; Horton, R. M., Hunt, H. D., Ho, S. N., Pullen, J. K. & Pease, L. R. Gene 77, 61-68.

### SUMMARY OF THE INVENTION

According to an aspect of some embodiments of the present invention there is provided a method of diagnosing a disease in a subject in need thereof, the method comprising:

(a) linking a plurality of non-contiguous DNA fragments from a sample of the subject to generate a polynucleotide product, the linking being effected by contacting the plurality of non-contiguous DNA fragments with a multiplex overlap-extension primer mix under conditions that allow simultaneous linkage of the DNA fragments and amplification of the polynucleotide product, wherein the primer mix comprises two flanking primers and at least one linker primer; and

(b) determining a sequence of the polynucleotide product, wherein the sequence is indicative of the disease.

According to an aspect of some embodiments of the present invention there is provided a method of linking a plurality of non-contiguous fragments of DNA to generate a single polynucleotide product, the method comprising contacting the fragments with a multiplex overlap-extension primer mix, the mix comprising two flanking primers and a number of linker primers being one less than a number of the fragments, under conditions which allow simultaneous linking of the fragments and amplification of the single polynucleotide product, thereby generating the single polynucleotide product.

According to an aspect of some embodiments of the present invention there is provided a method of linking a plurality of non-contiguous fragments of non-transcribed DNA to generate a single polynucleotide product, the method comprising contacting the fragments with a multiplex overlap-extension primer mix under conditions which allow simultaneous linking of the fragments and amplification of the single polynucleotide product, wherein the primer mix comprises two flanking primers and at least one linker primer, thereby generating the single polynucleotide product.

According to an aspect of some embodiments of the present invention there is provided a kit for linking a plurality of non-contiguous fragments of DNA to generate a polynucleotide product, the kit comprising at least two flanking primers and at least one linker primer wherein at least two of the non-contiguous fragments of DNA comprise a nucleic acid sequence which is indicative of a disease.

According to some embodiments of the invention, at least two of the non-contiguous fragments of DNA comprise a nucleic acid sequence which is indicative of a disease.

According to some embodiments of the invention, the method further comprises fragmenting DNA of the sample prior to step (a) so as to generate the plurality of non-contiguous DNA fragments.

According to some embodiments of the invention, the fragmenting is effected using a restriction enzyme.

According to some embodiments of the invention, the polynucleotide product is no longer than 1000 base pairs.

According to some embodiments of the invention, the plurality of non-contiguous fragments comprises two fragments.

According to some embodiments of the invention, the plurality of non-contiguous fragments comprises three fragments.

According to some embodiments of the invention, the multiplex overlap-extension primer mix comprises two flanking primers and at least two linker primers.

According to some embodiments of the invention, a concentration of the at least one linker primer is lower than a concentration of the two flanking primers.

According to some embodiments of the invention, the determining a sequence is effected using a chain termination method.

According to some embodiments of the invention, the method further comprises informing the subject of the results of the diagnosing.

According to some embodiments of the invention, the method further comprises performing additional diagnostic tests so as to corroborate the results of the diagnosing.

According to some embodiments of the invention, the DNA comprises non-transcribed DNA.

According to some embodiments of the invention, the subject is a fetus.

According to some embodiments of the invention, the sample comprises amniotic fluid.

According to some embodiments of the invention, the DNA comprises non-transcribed DNA.

According to some embodiments of the invention, a concentration of the linker primers is lower than a concentration of the flanking primers.

According to some embodiments of the invention, the kit further comprises a DNA polymerase enzyme.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C illustrate the generation of a chimeric DNA fragment by single step PCR reaction. (A) A Schematic diagram illustrating the logic underlining the design of a single step PCR ligation reaction of two DNA fragments. (B) A practical example for PCR ligation of two DNA fragments: the ubiquitin binding proteasomal subunit RPN10 (I—SEQ ID NO: 21) and ubiquitin (II—SEQ ID NO: 20). (C) PCR recombination of RPN10 and ubiquitin was preformed as described above while inserting one, two or three nucleotides between the two genes.

FIG. 1D is a cartoon illustrating the alignment of the linker primer with the two PCR templates.

FIGS. 2A-B illustrate three piece ligation by single step PCR recombination (A) A Schematic diagram describing the recombination of three segments of the yeast proteasomal subunits RPN10 (IV—SEQ ID NO: 23), RPT5 (V—SEQ ID NO: 24) and RPT2 (VI—SEQ ID NO: 25). (B) Analysis of the PCR recombination reaction on 1% agarose gel complimented with Ethidium Bromide demonstrating the formation of the full length three piece recombination products.

FIGS. 3A-B illustrate the calibration of the optimal concentration of linker-primer. Linker induced PCR recombination of RPN10-UBI1 (SEQ ID NO: 33) gene fragments in the presence of increasing concentrations of linker primer. (A) Reaction products separated on 1% agarose gel. (B) Intensity of the desired PCR product visualized by Ethidium bromide plotted as a function of the linker-primer concentrations. Note that the concentrations are given in logarithmic scale. The optimal primer-linker concentration was found to be about 2 nM.

FIG. 4 is a cartoon illustrating the alignment of the linker primer with the three PCR templates.

FIGS. 5A-B illustrate direct ligation of internal gene fragments by linker induced overlap recombination. Four linker-primers and two flanking primers were used to allow single step PCR ligation of fragments in which the desired recombination site resides in the internal sequence of the templates. (A) A carton representing the rational and the progression of the PCR recombination reaction. (B) agarose gel stained by Ethidium-bromide staining to visualized the reaction products.

FIG. 5C is a cartoon illustrating the alignment of the four linker primers with the three PCR templates.

FIGS. 6A-B illustrate ligation of the three fragments as described in FIGS. 5A-B using the classical two step overhang PCR recombination. FIG. 5A: Initially, the three fragment to be recombined were amplified by PCR (PCR reaction I). These PCR products were then used in a second PCR reaction (PCR reaction II) to generate the recombination product. FIG. 5B: agarose gel stained by Ethidium-bromide staining to visualized the reaction products—accusly FIG. 5A-B is a single PCR reaction in which the first part is what happened in the first few cycles and the second is what happened later in this reaction.

FIGS. 6C-D illustrate ligation of the three fragments described in FIGS. 6A-B using 2 flanking primers and 2 linker primers.

FIGS. 7A-B illustrate direct cloning of a desired gene into a plasmid by single step PCR. (A): pET 22-E2-S (IX—SEQ ID NO: 28) and the Active Site Loop (ASL) of E2-25K (VIII—SEQ ID NO: 27) served as temples for the amplification of a linear recombination products in which the native ASL of E2-S was swapped by the ASL of E2-25K (X—SEQ ID NO: 29). (B): The PCR products were separated on agarose gel, side by side with the initial templates. The gel was visualized by Ethidium bromide.

FIG. 7C is a cartoon illustrating the alignment of the linker primer with the two templates (E2-25K active loop, E2-S in pET22 plasmid).

FIGS. 8A-B illustrate direct cloning of a desired gene into a plasmid by single step PCR. (A): Ks (XI—SEQ ID NO: 30) and RPN10 (I—SEQ ID NO: 21) DNA fragment served as templates for the amplification of linear recombination products. (B): the PCR products of the reaction were separated on agarose gel, side by side with the initial templates. The gel was visualized by Ethidium bromide.

FIG. 8C is a cartoon illustrating the alignment of the linker primer with the two templates (KS+ Plasmid, Rpn10 PCR).

FIGS. 9A-B illustrates recombining DNA fragment from genomic source. Two linker-primers (f & g) and two flanking primers (a & s) were used to fused RPN10 (Chr. VII) and RPT5 (Chr. XV) utilizing yeast genomic DNA as template. FIG. 9A, visualization of the PCR reaction product separated on 1% agarose stained by Ethidium bromide. FIG. 9B, schematic presentation of the reaction.

### DESCRIPTION OF SPECIFIC EMBODIMENTS OF THE INVENTION

The present invention, in some embodiments thereof, relates to methods of diagnosing a disease or susceptibility thereto based on DNA sequencing.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Analysis of multiple DNA sequences has become necessary for the diagnosis of a particular disease as more and more mutations/variations are found to be disease associated.

In a classical multiplex PCR reaction, different fragments are amplified in a single tube, simply by adding all pairs of amplicon-specific primers to a reaction mixture.

The present inventors have now conceived of a different approach—namely the Linker-Induced Overlapping Recombination PCR method—enabling the promotion of an endonuclease-independent, single-step, PCR procedure to aid in disease diagnosis. The method allows for amplification of a DNA product which comprises a multitude of linked fragments, each fragment being indicative of a disease. By combining just diagnostically relevant fragments into one polynucleotide fragment, the amount of sequencing may be significantly reduced. The method may be particularly applicable to prenatal genetic testing whereby a multitude of diseases can be detected and diagnosed by a single reaction.

The underlying rational is based on a hierarchical PCR reaction in which by using an additional primer in a limited concentration an intermediate product is generated to a limited amount. The intermediate product then serves both as template and primer for the consecutive reaction that produces the desired end-product which is then amplified by flanking primers. Although all the ingredients are present in the PCR tube at onset of the reaction, the process proceeds through predefined steps guided by the concentration and complimentarity of the different primers included.

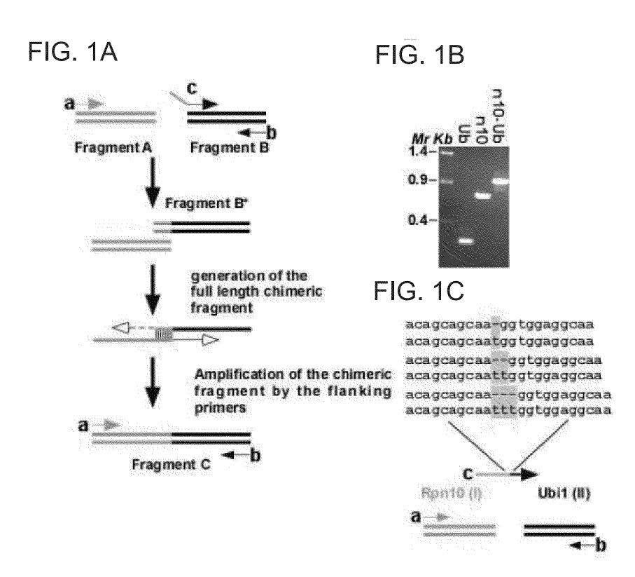

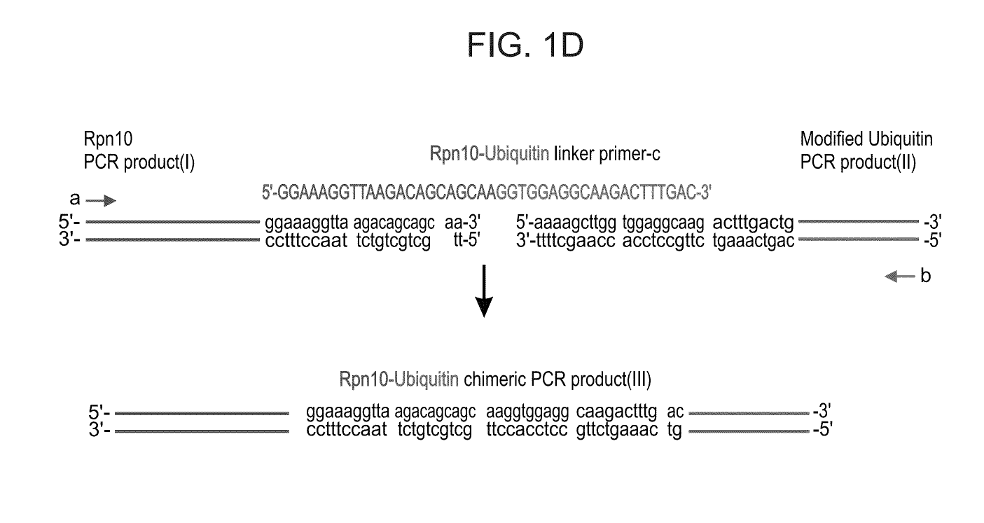

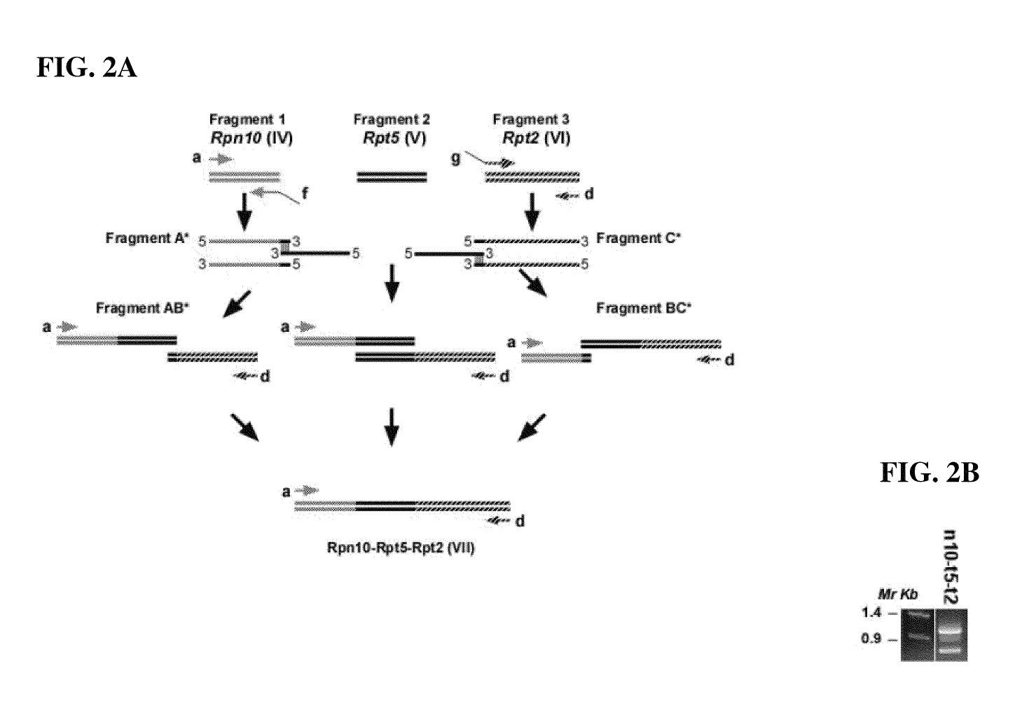

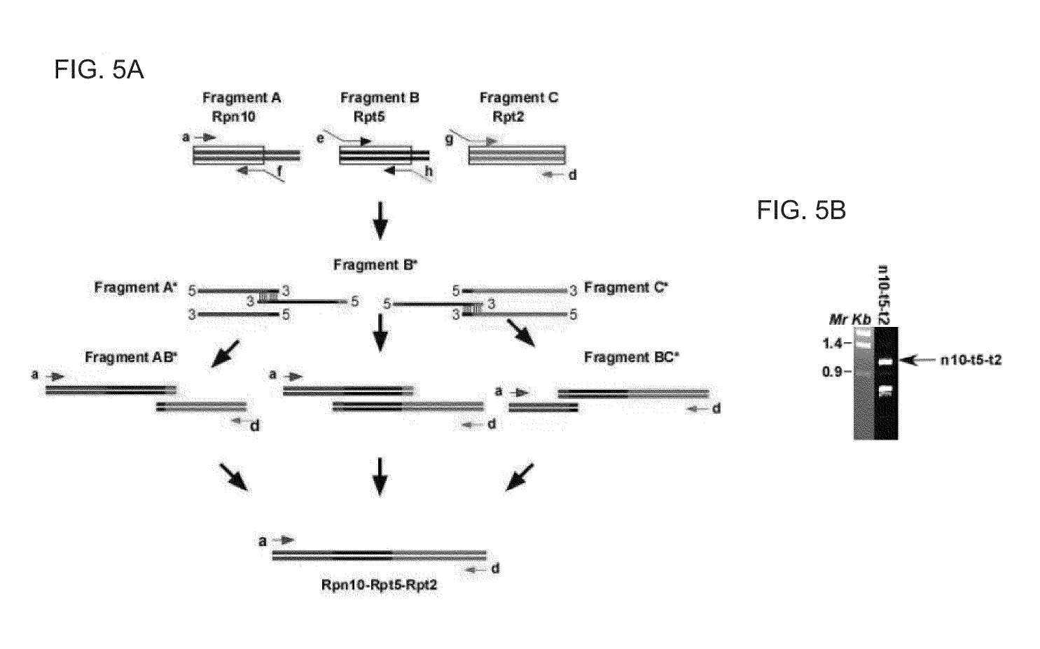

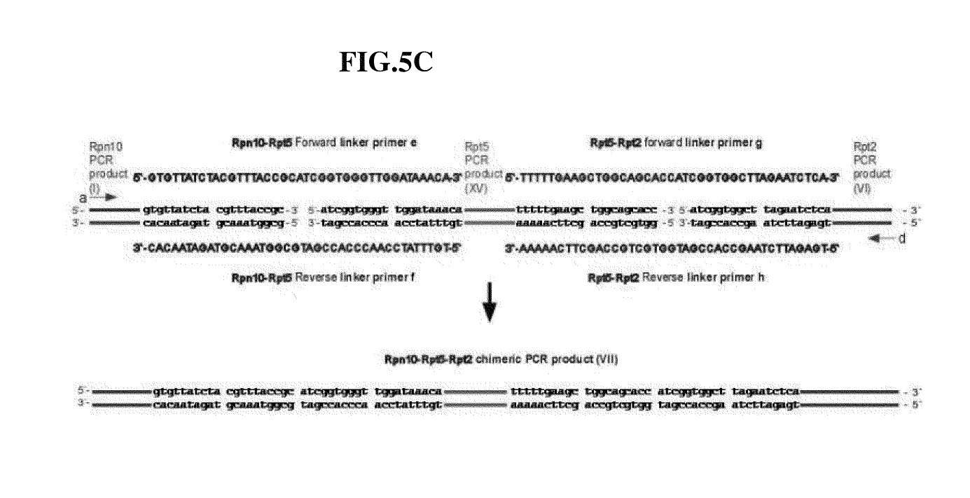

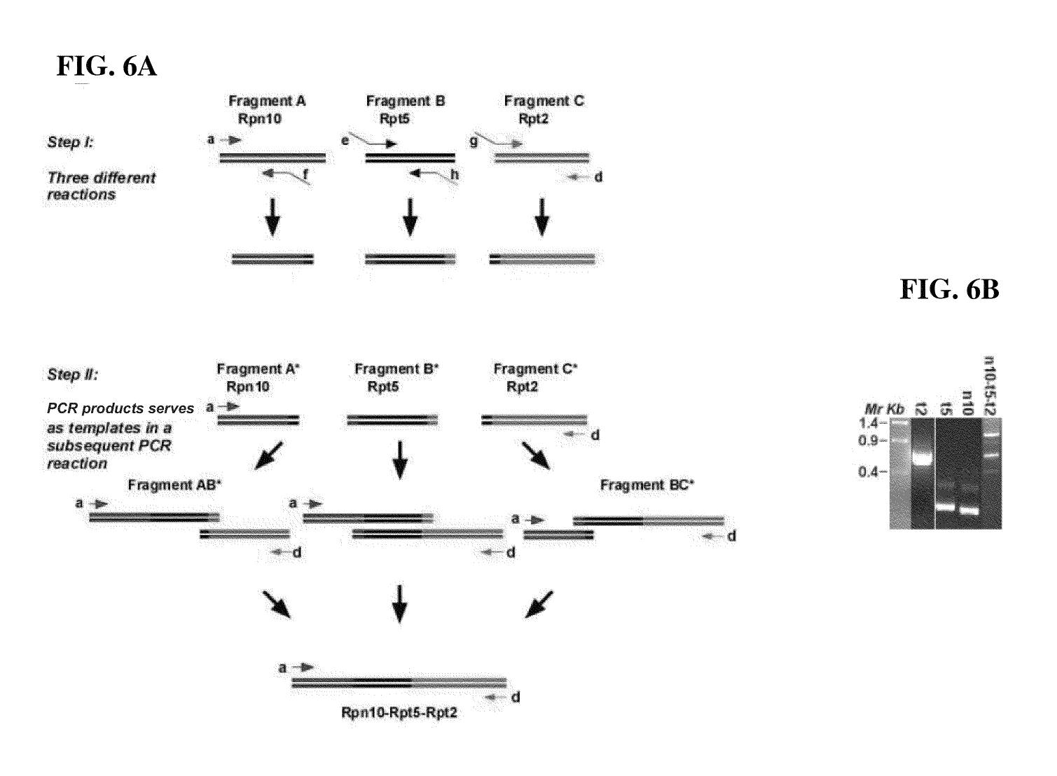

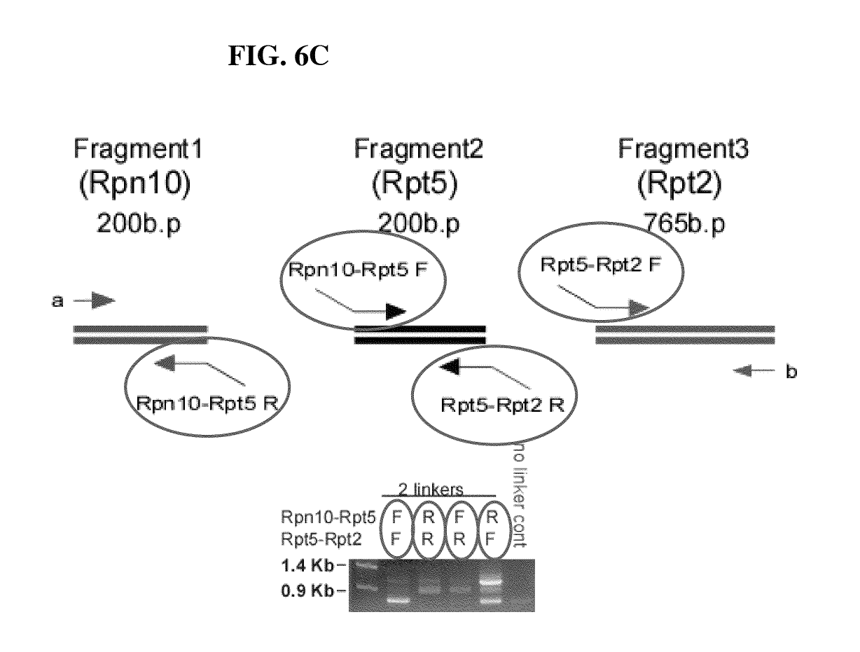

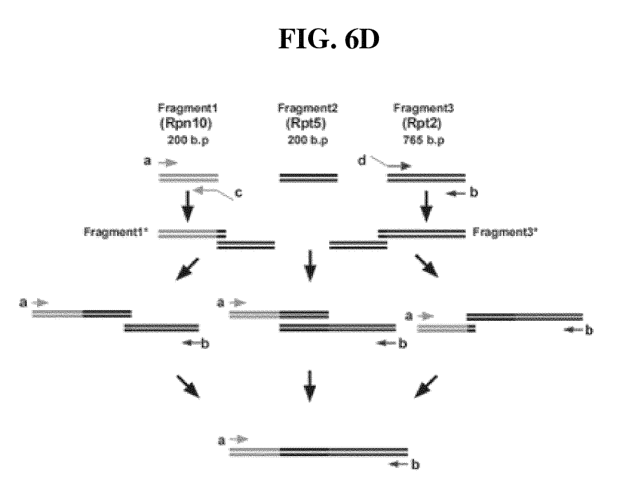

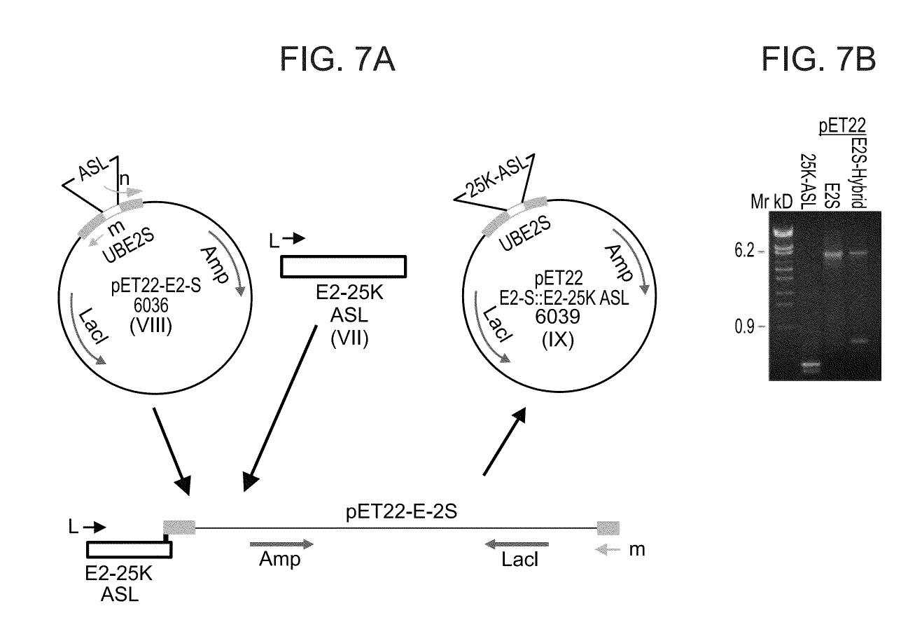

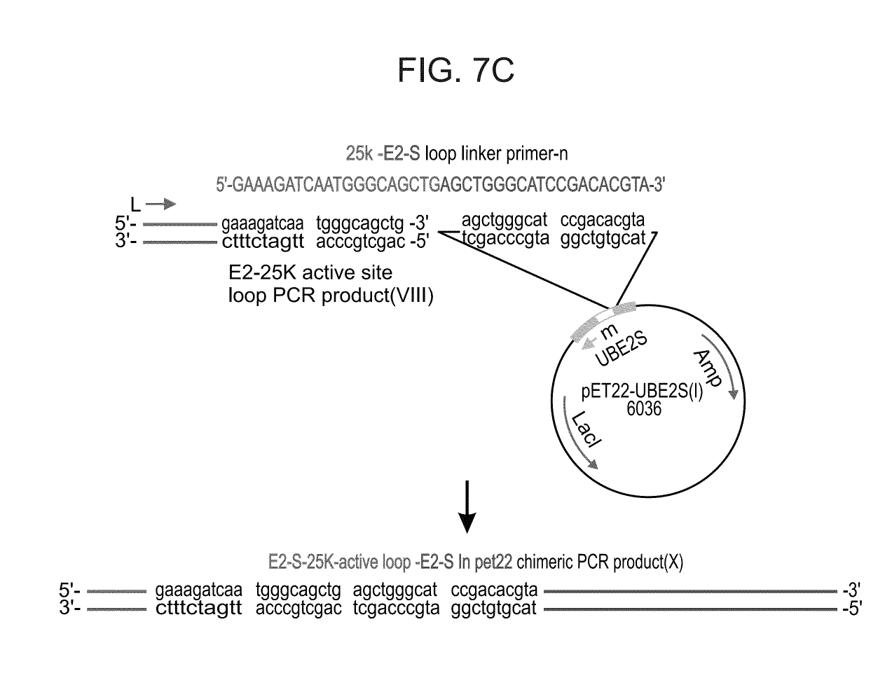

The method was successfully performed for the linkage of two (FIGS. 1A-C) and three (FIGS. 2A-B, FIGS. 5A-B and FIGS. 6A-B) fragments. In addition, the present inventors showed that the method can be used to simultaneously amplify and insert a DNA fragment into a plasmid, thereby significantly simplifying the cloning procedure (FIGS. 7A-B, 8A-C and 9A-B).

Thus, according to an aspect of the present invention, there is provided a method of diagnosing a disease in a subject in need thereof, the method comprising:

(a) linking a plurality of non-contiguous DNA fragments from a sample of the subject to generate a polynucleotide product, the linking being effected by contacting the plurality of non-contiguous DNA fragments with a multiplex overlap-extension primer mix under conditions that allow simultaneous linkage of the DNA fragments and amplification of the polynucleotide product, wherein the primer mix comprises two flanking primers and at least one linker primer; and

(b) determining a sequence of the polynucleotide product, wherein the sequence is indicative of the disease.

As used herein, the phrase “diagnosing a disease” refers to determining a presence of a disease, determining if the subject is a carrier for a particular disease, determining a predisposition to a disease, classifying a disease, determining a severity of disease (grade or stage), monitoring disease progression, forecasting an outcome of the disease and/or prospects of recovery.

The present invention contemplates diagnosing any disease which is associated with a change (i.e. mutation) in a nucleic acid sequence of a DNA of a subject as compared to the wild-type DNA.

As used herein, the term “wild-type” refers to the most common polynucleotide sequence or allele for a certain gene or non-coding sequence in a population. Generally, the wild-type DNA will be obtained from normal (non-diseased) cells.

As used herein, the term “mutant” refers to a nucleotide change (i.e., a single or multiple nucleotide substitution, deletion, or insertion) in a nucleic acid sequence. A nucleic acid which bears a mutation has a nucleic acid sequence (mutant allele) that is different in sequence from that of the corresponding wild-type polynucleotide sequence. Preferably, a mutation in DNA of a subject will contain between 1 and 10 nucleotide sequence changes. The mutant DNA may have 50%, 60%. 70%, 80%, 85%, 90%, 91%, 92%, 93%, 94%, 95%, 96%, 97%, 98%, 99% or more homology to the wild-type DNA.

Changes in the nucleic acid sequence of a DNA of a subject may be the result of natural or artificial (e.g., chemical carcinogens) deletions, additions, or substitutions of nucleotides.

An exemplary change in the nucleic acid sequence of DNA is one that takes the form of short tandem repeats (STRs) that include tandem di-, tri- and tetra-nucleotide repeated motifs. These tandem repeats are also referred to as variable number tandem repeat (VNTR) polymorphisms. VNTRs have been used in identity and paternity analysis (U.S. Pat. No. 5,075,217; Armour et al., FEBS Lett. 307:113-115 (1992); Horn et al., WO 91/14003; Jeffreys, EP 370,719), and in a large number of genetic mapping studies.

Another exemplary change in the nucleic acid sequence of DNA is one which takes the form of single nucleotide variations between individuals of the same species. Such polymorphisms are far more frequent than RFLPs, STRs (short tandem repeats) and VNTRs (variable number tandem repeats). Some single nucleotide polymorphisms occur in protein-coding sequences, in which case, one of the polymorphic forms may give rise to the expression of a defective or other variant protein and, potentially, a genetic disease. Other single nucleotide polymorphisms occur in noncoding regions. Some of these polymorphisms may also result in defective protein expression (e.g., as a result of defective splicing).

Other mutations include somatic mutations. Somatic mutations are alterations in DNA that occurs after conception. Somatic mutations can occur in any of the cells of the body except the germ cells (sperm and egg) and therefore are not passed on to children. These alterations can (but do not always) lead to cancer or other diseases.

Exemplary diseases which may be diagnosed include, but are not limited to heart disease, cancer, endocrine disorders, immune disorders, neurological disorders, musculoskeletal disorders, ophthalmologic disorders, genetic abnormalities, trisomies, monosomies, transversions, translocations, skin disorders and familial diseases.

The method of the invention is especially useful in prenatal genetic testing of parents and child. The method may be used for simultaneous diagnosis of a multitude of diseases. Examples of some of such diseases include, but are not limited to those listed herein below.

Achondroplasia Adrenoleukodystrophy, X-Linked Agammaglobulinemia, X-Linked Alagille Syndrome Alpha-Thalassemia X-Linked Mental Retardation Syndrome Alzheimer Disease Alzheimer Disease, Early-Onset Familial Amyotrophic Lateral Sclerosis Overview Androgen Insensitivity Syndrome Angelman Syndrome Ataxia Overview, Hereditary Ataxia-Telangiectasia Becker Muscular Dystrophy (also The Dystrophinopathies) Beckwith-Wiedemann Syndrome Beta-Thalassemia Biotimidase Deficiency Branchiootorenal Syndrome BRCA1 and BRCA2 Hereditary Breast/Ovarian Cancer Breast Cancer CADASIL Canavan Disease Cancer Charcot-Marie-Tooth Hereditary Neuropathy Charcot-Marie-Tooth Neuropathy Type 1 Charcot-Marie-Tooth Neuropathy Type 2 Charcot-Marie-Tooth Neuropathy Type 4 Charcot-Marie-Tooth Neuropathy Type X Cockayne Syndrome Colon Cancer Contractural Arachnodactyl), Congenital Craniosynostosis Syndromes (FGFR-Related) Cystic Fibrosis Cystinosis Deafness and Hereditary Hearing Loss DRPLA (Dentatorubral-Pallidoluysian Atrophy) DiGeorge Syndrome (also 22q11 Deletion Syndrome) Dilated Cardiomyopathy, X-Linked Down Syndrome (Trisomy 21) Duchenne Muscular Dystrophy (also The Dystrophinopathies) Dystonia, Early-Onset Primary (DYT1) Dystrophinopathies, The Ehlers-Danlos Syndrome, Kyphoscoliotic Form Ehlers-Danlos Syndrome, Vascular Type Epidermolysis Bullosa Simplex Exostoses, Hereditary Multiple Facioscapulohumeral Muscular Dystrophy Factor V Leiden Thrombophilia Familial Adenomatous Polyposis (FAP) Familial Mediterranean Fever Fragile X Syndrome Friedreich Ataxia Frontotemporal Dementia with Parkinsonism-17 Galactosemia Gaucher Disease Hemochromatosis, Hereditary Hemophilia A Hemophilia B Hemorrhagic Telangiectasia, Hereditary Hearing Loss and Deafness, Nonsyndromic, DFNA3 (Connexin 26) Hearing Loss and Deafness, Nonsyndromic, DFNB1 (Connexin 26) Hereditary Spastic Paraplegia Hermansky-Pudlak Syndrome Hexosaminidase A Deficiency (also Tay-Sachs) Huntington Disease Hypochondroplasia Ichthyosis, Congenital, Autosomal Recessive Incontinentia Pigmenti Kennedy Disease (also Spinal and Bulbar Muscular Atrophy) Krabbe Disease Leber Hereditary Optic Neuropathy Lesch-Nyhan Syndrome Leukemias Li-Fraumeni Syndrome Limb-Girdle Muscular Dystrophy Lipoprotein Lipase Deficiency, Familial Lissencephaly Marfan Syndrome MELAS (Mitochondrial Encephalomyopathy, Lactic Acidosis, and Stroke-Like Episodes) Monosomies Multiple Endocrine Neoplasia Type 2 Multiple Exostoses, Hereditary Muscular Dystrophy, Congenital Myotonic Dystrophy Nephrogenic Diabetes Insipidus Neurofibromatosis 1 Neurofibromatosis 2 Neuropathy with Liability to Pressure Palsies, Hereditary Niemann-Pick Disease Type C Nijmegen Breakage Syndrome Norrie Disease Oculocutaneous Albinism Type 1 Oculopharyngeal Muscular Dystrophy Ovarian Cancer Pallister-Hall Syndrome Parkin Type of Juvenile Parkinson Disease Pelizaeus-Merzbacher Disease Pendred Syndrome Peutz-Jeghers Syndrome Phenylalanine Hydroxylase Deficiency Prader-Willi Syndrome PROP1-Related Combined Pituitary Hormone Deficiency (CPHD) Prostate Cancer Retinitis Pigmentosa Retinoblastoma Rothmund-Thomson Syndrome Smith-Lemli-Opitz Syndrome Spastic Paraplegia, Hereditary Spinal and Bulbar Muscular Atrophy (also Kennedy Disease) Spinal Muscular Atrophy Spinocerebellar Ataxia Type 1 Spinocerebellar Ataxia Type 2 Spinocerebellar Ataxia Type 3 Spinocerebellar Ataxia Type 6 Spinocerebellar Ataxia Type 7 Stickler Syndrome (Hereditary Arthroophthalmopathy) Tay-Sachs (also GM2 Gangliosidoses) Trisomies Tuberous Sclerosis Complex Usher Syndrome Type I Usher Syndrome Type II Velocardiofacial Syndrome (also 22q11 Deletion Syndrome) Von Hippel-Lindau Syndrome Williams Syndrome Wilson Disease X-Linked Adrenoleukodystrophy X-Linked Agammaglobulinemia X-Linked Dilated Cardiomyopathy (also The Dystrophinopathies) X-Linked Hypotonic Facies Mental Retardation Syndrome

As mentioned, the method is effected by linking a plurality of non-contiguous DNA fragments from a sample of the subject to generate a polynucleotide product.

DNA may be obtained from any biological sample including but not limited to blood sample, serum sample, amniotic fluid sample, plasma sample, urine sample, spinal fluid, lymphatic fluid, semen, vaginal secretion, ascitic fluid, saliva, mucosa secretion, peritoneal fluid, fecal sample, body exudates, breast fluid, lung aspirates, cells, tissues, individual cells or extracts of the such sources that contain the nucleic acid of the same, and subcellular structures such as mitochondria.

The DNA may be genomic DNA (e.g. non-transcribed DNA, coding DNA or non-coding DNA) or cDNA (reverse transcribed DNA). A reverse transcription (RT) reaction may be performed with an enzyme containing reverse transcriptase activity resulting in the generation of cDNA from total RNA, mRNA or target specific RNA from an isolated single cell. Primers which can be utilized for the reverse transcription are for example oligo-dT primers, random hexamers, random decamers, other random primers, or primers that are specific for the nucleotide sequences of interest.

The non-contiguous DNA fragments of this aspect of the present invention may comprise sequences of the same gene or different genes. Further, the non-contiguous DNA fragments of this aspect of the present invention may comprise sequences of the same chromosome or different chromosomes. It will be appreciated that the distance between the DNA fragments of interest in their natural location (e.g. on a particular gene or chromosome) need not be considered when selecting the particular DNA fragments to be located. Typically, the DNA fragments are not closer than 500 base pairs away from each other in their natural environment.

According to one embodiment, the DNA fragments comprise sequences of genes involved in a given pathway or disease. According to another embodiment, the DNA fragments comprise sequences of genes belonging to a certain class of proteins. According to yet another embodiment, each DNA fragment comprises a putative site for a single nucleotide polymorphisms (SNP).

Each DNA fragment is typically less than about 5000 base pair (e.g. less than about 1000 base pairs, 500 base pairs, less than 400 base pairs, less than 300 base pairs, less than 200 base pairs or less than 100 base pairs).

Typically, the DNA fragments are selected such that at least two comprise a nucleic acid sequence which is indicative of a disease. It will be appreciated that the nucleic acid sequences may be indicative of one particular disease or different diseases.

The number of DNA fragments that may be linked according to this aspect of the present invention is typically between 2 and 10 and more typically between 2 and 5—for example, 2, 3 or 4.

The sample may be processed before the method is carried out, for example DNA purification may be carried out following the extraction procedure. The DNA in the sample may be cleaved either physically or chemically (e.g. using a suitable enzyme). Processing of the sample may involve one or more of: filtration, distillation, centrifugation, extraction, concentration, dilution, purification, inactivation of interfering components, addition of reagents, and the like.

DNA fragments may be prepared using any number of methods well known in the art including but not limited to enzymatic digestion, manual shearing, and sonication. For example, the DNA can be digested with one or more restriction enzymes that have a recognition site, and especially an eight base or six base pair recognition site, which is not present in the loci of interest.

The term “linking” refers to the joining of DNA fragments into a single product. The linkage is preferably a nucleotide phosphodiester linkage. However, linkage can also be obtained by different chemical cross linking procedures.

As mentioned, the linking is effected by contacting the plurality of non-contiguous DNA fragments with a multiplex overlap-extension primer mix under conditions that allow simultaneous linkage of the DNA fragments and amplification of the polynucleotide product.

The method of this aspect of the present invention is effected by performing a multiplex molecular amplification reaction, using a primer mix which comprises two flanking primers and at least one linker primer.

The term “multiplex molecular amplification” describes the simultaneous amplification of two or more target sequences in the same reaction. Suitable amplification methods include the polymerase chain reaction (PCR) (U.S. Pat. No. 4,683,202), ligase chain reaction (LCR), (Wu and Wallace, 1989, Genomics 4, 560-9), strand displacement amplification (SDA) technique (Walker et al., 1992, Nucl. Acids Res. 20, 1691-6), self-sustained sequence replication (Guatelli et al., 1990, Proc. Nat. Acad. Sci. U.S.A., 87, 1874-8) and nucleic acid based sequence amplification (NASBA) (Compton J., 1991, Nature 350, 91-2). The latter two amplification methods involve isothermal reactions based on isothermal transcription, which produce both single stranded RNA (ssRNA) and double stranded DNA (dsDNA).

One feature of the present invention reduces the number of tubes necessary to amplify the nucleotide sequences of interest, utilizing a variant of PCR in which two or more target sequences are amplified and linked simultaneously in the same tube, by including more than one set of primers.

The PCR (or polymerase chain reaction) technique is well-known in the art and has been disclosed, for example, in K. B. Mullis and F. A. Faloona, Methods Enzymol., 1987, 155: 350-355 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). RNA target sequences may be amplified by reverse transcribing the mRNA into cDNA, and then performing PCR(RT-PCR), as described above. Alternatively, a single enzyme may be used for both steps as described in U.S. Pat. No. 5,322,770.

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 40.

The denaturation step of a PCR cycle generally comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually from about 93° C. to about 96° C. for a period of time ranging from about 3 to about 120 seconds, usually from about 5 to about 30 seconds.

Following denaturation, the reaction mixture is subjected to conditions sufficient for primer annealing to template DNA present in the mixture. The temperature to which the reaction mixture is lowered to achieve these conditions is usually chosen to provide optimal efficiency and specificity, and generally ranges from about 50° C. to about ° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

Following annealing of primer to template DNA or during annealing of primer to template DNA, the reaction mixture is subjected to conditions sufficient to provide for polymerization of nucleotides to the primer's end in a such manner that the primer is extended in a 5′ to 3′ direction using the DNA to which it is hybridized as a template, (i.e., conditions sufficient for enzymatic production of primer extension product). To achieve primer extension conditions, the temperature of the reaction mixture is typically raised to a temperature ranging from about 65° C. to about 75° C., usually from about 67° C. to about 73° C., and maintained at that temperature for a period of time ranging from about 15 seconds to about 20 minutes, usually from about 30 seconds to about 5 minutes.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

Amplification products obtained using primers of the present invention may be detected using agarose gel electrophoresis and visualization by ethidium bromide staining and exposure to ultraviolet (UV) light or by sequence analysis of the amplification product.

Primers of the invention may be prepared by any of a variety of methods (see, for example, J. Sambrook et al., “Molecular Cloning: A Laboratory Manual”, 1989, 2.sup.nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5.sup.th Ed., John Wiley & Sons: Secaucus, N.J.). For example, oligonucleotides may be prepared using any of a variety of chemical techniques well-known in the art, including, for example, chemical synthesis and polymerization based on a template as described, for example, in S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066.

For example, primers may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of the primers of the invention, where necessary or desirable, may be carried out by any of a variety of methods well-known in the art. Purification of oligonucleotides is typically performed either by native acrylamide gel electrophoresis, by anion-exchange HPLC as described, for example, by J. D. Pearson and F. E. Regnier (J. Chrom., 1983, 255: 137-149) or by reverse phase HPLC (G. D. McFarland and P. N. Borer, Nucleic Acids Res., 1979, 7: 1067-1080).

The sequence of the primers can be verified using any suitable sequencing method including, but not limited to, chemical degradation (A. M. Maxam and W. Gilbert, Methods of Enzymology, 1980, 65: 499-560), matrix-assisted laser desorption ionization time-of-flight (MALDI-TOF) mass spectrometry (U. Pieles et al., Nucleic Acids Res., 1993, 21: 3191-3196), mass spectrometry following a combination of alkaline phosphatase and exonuclease digestions (H. Wu and H. Aboleneen, Anal. Biochem., 2001, 290: 347-352), and the like.

Generally, the components needed for the single-step multiplex overlap-extension reaction comprises the DNA fragments, an enzyme with DNA polymerase activity, deoxynucleoside triphosphate mix (dNTP mix comprising dATP, dCTP, dGTP and dTTP) and a multiplex overlap extension primer mix. In order to perform multiplex overlap-extension PCR on two fragments, the presence of at least three primers are required (a multiplex primer mix), where at least one primer is equipped with an overlap-extension tail (i.e. linking primer). The overlap-extension tails enable the linkage of the products. Such a primer mix is called a multiplex overlap-extension primer mix. The multiplex overlap-extension PCR, differ from conventional overlap-extension PCR in that the sequences to be linked are generated simultaneously in the same tube, thereby providing immediate linkage of the target sequences during amplification, without any intermediate purification. Further, conventional overlap-extension PCR requires a separate linking PCR reaction either with an outer primer set or a nested primer set in order to generate the linked product (Horton, R. M. et al. 1989. Gene 77, 61-68). Such an additional amplification step is optional in the multiplex overlap-extension PCR of the present invention.

According to one embodiment, the linkage of two DNA fragments is effected using 1 linking primer and 2 flanking primers (as illustrated in FIGS. 1A-B).

The first flanking primer hybridizes to the 5′ region of the sense strand of the first fragment, whereas the second flanking primer hybridizes to the 3′ end of the antisense strand of the second fragment. The linker primer is selected such that its nucleotide sequence reflects the desired junction between the two DNA fragments. Specifically, the initial nucleotides of the linker primer are identical to the 3′ region of the sense strand of the first fragment, while the remaining nucleotides are derived from the 5′ of the sense strand of the second fragment.

According to one embodiment, the linkage of three DNA fragments is effected using 2 linking primer and 2 flanking primers (as illustrated in FIGS. 2A-B).

Other contemplated configurations of linking primers and flanking primers are described in the Examples section below.

The design of the flanking primers generally should observe known primer design rules such as minimizing primer dimerization, hairpin formation and non-specific annealing. Further, multiple G or C nucleotides as the 3′ bases are to be avoided when possible. The melting temperature (Tm) of the gene-specific regions in a primer set should preferably be equal to each other plus/minus 5° C. For example Tm values between 45° C. and 75° C. may be desirable and Tm values of about 60° C. are optimal for most applications. Advantageously, the initial primer design can be aided by computer programs developed for this task.

Design of the linking primer is dependent on sequence features such as length, relative GC content (GC %), presence of restriction sites, palindromes, melting temperature, the gene-specific part to which they are coupled etc. The length of the overlap-extension tails should be between 8 and 75 nucleotides long, preferably they are from 15 to 40 nucleotides long. Even more preferred they are from 22 to 28 nucleotides long. The use of very long overlap-extension tails (50 to 75 nucleotides) could favor the linkage of the products produced by each primer set. However, the proportion between the length of the overlap-extension tail and the gene-specific region probably will need to be adjusted when using very long overlap-extension tails. The GC % preference is dependent on the length of the overlap-extension tail. Since shorter tails have a shorter area where they are complementary they need a higher GC % to strengthen the interaction than longer tails. Other principles of primer design should likewise be observed, e.g. primer dimerization and hairpin formation should be minimized. Neither shall they engage in false priming. Further, it is known that Taq DNA polymerase often adds an adenosine (A) at the 3′ end of the newly synthesized DNA strand, and this can be accommodated for in overlap-extension tail design by enabling overlap-extension tails to accommodate 3′ non-template A addition.

It will be appreciated that the primers of this aspect of the present invention need not reflect the exact sequence of the target nucleic acid sequences (i.e. need not be fully complementary), but must be sufficiently complementary to hybridize with their target sequences under the particular experimental conditions. Accordingly, the sequence of the primer typically has at least 70% homology, preferably at least 80%, 90%, 95%, 97%, 99% or 100% homology, for example over a region of at least 13 or more contiguous nucleotides with the target DNA fragments.

The parameters of the procedure of this aspect of the present invention can be optimized on several parameters as detailed herein below:

a. Primer Concentration:

The concentration of the linking primers is preferably lower than the concentration of the flanking primers. Suitable ratios of linking primer: flanking primer include 1:10, 1:20, 1:50, 1:100, 1:150, 1:200, 1:250, 1:300, 1:400 and 1:500.

If one of the target sequences amplifies with a lower efficiency than the others, for example, as a result of a higher GC %, it may be possible to equalize the amplification efficacy. This may be done by using a higher concentration of the primer set which mediates amplification with low efficiency, or lowering the concentration of the other primer set.

Further, when using a large number of primers the total primer concentration might be an issue. The upper limit is determined experimentally by titration experiments. Such an upper limit of total oligonucleotide concentration influences the maximal concentration of individual primers. If the individual primer concentration is too low it is likely to cause a poor PCR sensitivity.

The quality of the oligonucleotide primers have also been found to be important for the multiplex overlap-extension PCR. HPLC-purified oligonucleotides, have produced the best results.

b. PCR Cycling Conditions:

Exemplary cycling conditions are provided in the Examples section herein below.

Problems with poor PCR sensitivity, for example due to low primer concentration or template concentration can be overcome by using a high number of thermal cycles. A high number of thermal circles constitute between 35 and 80 cycles, preferably around 40 cycles. Further, longer extension times can improve the multiplex overlap-extension PCR process.

c. Use of Adjuvants

Multiplex PCR reactions can be significantly improved by using a PCR additive, such as DMSO, glycerol, formamide, or betaine, which relax DNA, thus making template denaturation easier.

d. dNTP and MgCl₂

Deoxynucleoside triphosphate (dNTP) quality and concentration is important for the multiplex overlap-extension PCR. Optimal dNTP concentration is between 200 and 600 μM (e.g. 400 μM) of each dNTP (dATP, dCTP, dGTP and dTTP), above which the amplification is rapidly inhibited. dNTP stocks are sensitive to thawing/freezing cycles. After three to five such cycles, multiplex PCR often do not work well. To avoid such problems, small aliquots of dNTP can be made and kept frozen at −20° C.

Optimization of Mg²⁺ concentration is important since most DNA polymerases are magnesium-dependent enzymes. In addition to the DNA polymerase, the template DNA primers and dNTP's bind Mg²⁺. Therefore, the optimal Mg²⁺ concentration will depend on the dNTP concentration, template DNA, and sample buffer composition. If primers and/or template DNA buffers contain chelators such as EDTA or EGTA, the apparent Mg²⁺ optimum may be altered. Excessive Mg²⁺ concentration stabilizes the DNA double strand and prevents complete denaturation of DNA, which reduces yield. Excessive Mg²⁺ can also stabilize spurious annealing of primer to incorrect template sites, thereby decreasing specificity. On the other hand, an inadequate Mg.sup.2+ concentration reduces the amount of product.

A good balance between dNTP and MgCl²⁺ is approximately 200 to 400 μM dNTP (of each) to 1.5 to 3 mM MgCl₂ or MgSo₄.

e. PCR Buffer Concentration

Generally KCl based buffers may be used for multiplex overlap-extension PCR; however, buffers based on other components such as (NH₄).2SO₄, MgSO₄, Tris-HCl, or combinations thereof may also be optimized to function with the multiplex overlap-extension PCR.

Selection of the buffer is also dependent on the particular enzyme which is used in the reaction. Thus, for example when the enzyme is PFU, the PFU buffer composition is (10×Pfu Buffer: 200 mM Tris-HCl (pH 8.8 at 25° C.), 100 mM (NH₄)₂SO₄, 100 mM KCl, 1% (v/v) Triton X-100, 1 mg/ml BSA and 20 mM MgSO₄). When Phusion DNA polymerse is used may be as follows: 10× Phusion DNA Buffer: 200 mM Tris-HCl (pH 8.8 at 25° C.), 100 mM (NH₄)₂SO₄, 600 mM KCl, 1% (v/v) Triton X-100, 1 mg/ml BSA and 20 mM MgSO₄

Primer pairs involved in the amplification of longer products may work better at lower salt concentrations (e.g. 20 to 50 mM KCl), whereas primer pairs involved in the amplification of short products work better at higher salt concentrations (e.g. 80 to 100 mM KCl). Raising the buffer concentration to 2× instead of 1× may improve the efficiency of the multiplex reaction.

f. DNA Polymerase

The present invention is exemplified with Pfu polymerase. Alternatively, other types of heat-resistant DNA polymerases including, for example, taq, Phusion, Pwo, Tgo, Tth, Vent, Deep-vent may be used. Polymerases without or with 3′ to 5′ exonuclease activity may either be used alone or in combination with each other.

Following production of the polynucleotide product according to the method of this aspect of the present invention, its sequence is determined.

The term “sequencing” refers to any technique known in the art that allows the order of at least some consecutive deoxyribonucleotides in at least part of an amplification product. Some non-limiting examples of sequencing techniques include Sanger's dideoxy termination method and the chemical cleavage method of Maxam and Gilbert, including variations of those methods; sequencing by hybridization; sequencing by synthesis; and restriction mapping. In certain embodiments, sequencing comprises electrophoresis, including gel electrophoresis and capillary electrophoresis, including miniaturized capillary electrophoresis, and often comprising laser-induced fluorescence; sequencing by hybridization including bead array microarray hybridization; microfluidics (see, e.g., Paegel et al., Analyt. Chem. 74:5092-98, 2002); mass spectrometry (see, e.g., Koster et al., Nat. Biotechnol. 14:1123-28, 1996); single molecule detection, including fluorescence microscopy or a nanometer-scale pore or nanopore; or combinations thereof. In some embodiments, sequencing comprises direct sequencing, duplex sequencing, cycle sequencing, single base extension (SBE) sequencing, solid-phase sequencing, Simultaneous Bi-directional Sequencing (SBS), double ended sequencing (see, e.g., Published PCT Application No. WO 2004/070005 A2), or combinations thereof. In some embodiments, sequencing comprises asymmetric PCR or A-PCR. In some embodiments, sequencing comprises an extending enzyme comprising a first fluorescent reporter group, such as a FRET donor, and a NTP comprising a second fluorescent reporter group, such as a quencher (see, e.g., U.S. Published Patent Application No. US 2003/0064366 A1). In some embodiments, sequencing comprises detecting at least some amplification products using an instrument, for example but not limited to an ABI PRISM® 377 DNA Sequencer, an ABI PRISM® 310, 3100, 3100-Avant, 3730, or 3730xI Genetic Analyzer, an ABI PRISM® 3700 DNA Analyzer (all from Applied Biosystems), a microarray or bead array, a fluorimeter, or a mass spectrometer. In some embodiments, sequencing comprises incorporating a dNTP, including a dATP, a dCTP, a dGTP, a dTTP, a dUTP, a dITP, or combinations thereof and including dideoxyribonucleotide versions of dNTPs (e.g., ddATP, ddCTP, ddGTP, ddITP, ddTTP, and ddUTP), into an amplification product. In some embodiments, sequencing comprises a sequencing grade DNA-dependent DNA polymerase, for example but not limited to, AmpliTaq DNA polymerase CS or FS (Applied Biosystems); Sequenase or Thermo Sequenase (USB Corp.); and Sequencing Grade Taq DNA Polymerase (Promega). In some embodiments, sequencing comprises: a DNA-dependent DNA polymerase, for example but not limited to the Klenow fragment of E. coli DNA Pol I; an ATP sulfurylase, for example but not limited to a recombinant S. cerevisiae ATP sulfurylase, a luciferase, including firefly luciferase, or a sulfurylase-luciferase fusion protein (a non-limiting example of an enzymatically active mutant or variant of an ATP sulfurylase and of a luciferase; see, e.g., U.S. Patent Publication Nos. US 2003/0113747 A1 and US 2003/0119012 A1); and optionally, an apyrase. In some embodiments, a sequencing reaction comprises dATP.alpha.S, typically in place of dATP. In some embodiments, sequencing further comprises detecting light or fluorescence using, for example but not limited to a photodiode, a photomultiplier tube, a charge-coupled camera (CCD), a fluorimeter, a laser-scanner coupled with a detector, or combinations thereof.

Descriptions of exemplary sequencing techniques can be found in, among other places, McPherson, particularly in Chapter 5; Sambrook and Russell; Ausubel et al.; Siuzdak, The Expanding Role of Mass Spectrometry in Biotechnology, MCC Press, 2003, particularly in Chapter 7; Di Giusto and King, Nucl. Acids Res. 31:e7; Schena, Microarray Analysis, John Wiley & Sons, 2003, particularly in Chapter 13; BigDye® Terminator v 1.1 or v3.1 Cycle Sequencing Kit Protocols (Applied Biosystems P/N 4337036 or 4337035, respectively); Ronaghi, Genome Res. 11:3-11, 2001; Agah et al., Nucl. Acids Res. 32:e166, 2004; Kartalov and Quake, Nucl. Acids Res. 32:2873-79, 2004; Cheuk-Wai Kan et al., Electrophoresis 25:3564-88, 2004; and Rapley.

Comparing the sequences comprised in the polynucleotide product to known human sequences (e.g. a consensus sequence such as that published as part of the human genome) allows for the diagnosis of particular diseases.

On obtaining a result of the sequence the subject is typically informed. Additional diagnostic tests may also be performed so as to corroborate the results of the diagnosing.

The methods of this aspect of the present invention may be practiced by providing the reagents used in the methods in the form of kits. A kit preferably contains one or more of the following components: written instructions for the use of the kit, appropriate buffers, salts, DNA extraction detergents, primers, nucleotides, labeled nucleotides, 5′ end modification materials, and if desired, water of the appropriate purity, confined in separate containers or packages, such components allowing the user of the kit to extract the appropriate nucleic acid sample, and analyze the same according to the methods of the invention. The primers that are provided with the kit will vary, depending upon the purpose of the kit and the DNA that is desired to be tested using the kit. In preferred embodiments the kits contain primers that allows for the linkage of two fragments, both of the fragments providing valuable information for single or multiple disease diagnosis.

A kit can also be designed to detect a desired or variety of single nucleotide polymorphisms, especially those associated with an undesired condition or disease. For example, one kit can comprise, among other components, a set or sets of primers to amplify and link at least two fragments of interest both of which are associated with breast cancer. Another kit can comprise, among other components, a set or sets of primers for genes associated with a predisposition to develop type I or type II diabetes. Still, another kit can comprise, among other components, a set or sets of primers for genes associated with a predisposition to develop heart disease.

It will be appreciated that the presently described method for simultaneous linking and amplification of DNA fragments may be useful for other purposes besides disease diagnosis.

Thus for example, the method of the invention can be used to genotype microorganisms so as to rapidly identify the presence of a specific microorganism in a substance, for example, a food substance. In that regard, the method of the invention provides a rapid way to analyze food, liquids or air samples for the presence of an undesired biological contamination, for example, microbiological, fungal or animal waste material. The invention is useful for detecting a variety of organisms, including but not limited to bacteria, viruses, fungi, protozoa, molds, yeasts, plants, animals, and archaebacteria. The invention is useful for detecting organisms collected from a variety of sources including but not limited to water, air, hotels, conference rooms, swimming pools, bathrooms, aircraft, spacecraft, trains, buses, cars, offices, homes, businesses, churches, parks, beaches, athletic facilities, amusement parks, theaters, and any other facility that is a meeting place for the public.

The method of the invention can be used to test for the presence of many types of bacteria or viruses in blood cultures from human or animal blood samples.

The method of the invention can also be used to confirm or identify the presence of a desired or undesired yeast strain, or certain traits thereof, in fermentation products, e.g. wine, beer, and other alcohols or to identify the absence thereof.

The method of the invention can also be used to confirm or identify the relationship of a DNA of unknown sequence to a DNA of known origin or sequence, for example, for use in criminology, forensic science, maternity or paternity testing, archeological analysis, and the like.

The method the invention can also be used to determine the genotypes of plants, trees and bushes, and hybrid plants, trees and bushes, including plants, trees and bushes that produce fruits and vegetables and other crops, including but not limited to wheat, barley, corn, tobacco, alfalfa, apples, apricots, bananas, oranges, pears, nectarines, figs, dates, raisins, plums, peaches, apricots, blueberries, strawberries, cranberries, berries, cherries, kiwis, limes, lemons, melons, pineapples, plantains, guavas, prunes, passion fruit, tangerines, grapefruit, grapes, watermelon, cantaloupe, honeydew melons, pomegranates, persimmons, nuts, artichokes, bean sprouts, beets, cardoon, chayote, endive, leeks, okra, green onions, scallions, shallots, parsnips, sweet potatoes, yams, asparagus, avocados, kohlrabi, rutabaga, eggplant, squash, turnips, pumpkins, tomatoes, potatoes, cucumbers, carrots, cabbage, celery, broccoli, cauliflower, radishes, peppers, spinach, mushrooms, zucchini, onions, peas, beans, and other legumes.

The method of the invention may also be useful for analyzing genetic variations of an individual that have an effect on drug metabolism, drug interactions, and the responsiveness to a drug or to multiple drugs. The method of the invention is especially useful in pharmacogenomics.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Fusion of the Yeast (S. cerevisiae) Proteasomal Subunit RPN10 (n10) to a Ubiquitin (Ub) Gene

Materials and Methods

A PCR reaction was set up as detailed in Table 1, herein below.

TABLE 1

| component | amount | |
|---|---|---|
| Distilled water (dH2O) | 36.9 | μl |
| 10X PFU buffer | 5 | μl |
| 100% DMSO | 1.5 | μl |
| dNTPs (2.5 mM each NTP) | 4 | μl |
| RPN10 PCR 10 ng/μl | 0.2 | μl |
| Ubiquitin PCR 10 ng/μl | 0.2 | μl |
| Rpn10 Forward primer (a) 50 nmol/ml | 0.5 | μl |
| Ubiquitin Reverse primer (b) 50 nmol/ml | 0.5 | μl |
| RPN10-Ubiquitin linker primer (c) | 0.2 | μl |
| 500 pmol/ml | | |
| Pfu DNA polymerase (2.5 U/μl) | 1 | μl (2.5U) |
| Total reaction volume | 50 | μl |

The sequences of the reaction primers are provided in Table 2, herein below.

TABLE 2

| | | | SEQ |
|---|---|---|---|
| | | | ID |
| symbol | Name | Sequence | NO |
| a | Rpn10 Forward | ATGGTATTGGAAGCTACAGT | 1 |
| | primer | | |
| b | Ubiquitin Reverse | TTGTCGACTCAACCTCTCAAT | 2 |
| | primer | CTCAAGA | |
| c | Rpn10 ubiquitin | GGAAAGGTTAAGACAGCAGC | 3 |
| | linker | AAGGTGGAGGCAAGACTTTGAC | |

The PCR cycling parameters are set forth in Table 3, herein below.

TABLE 3

| | Number of | | | |
|---|---|---|---|---|
| segment | cycles | temperature | duration | |
| 1 | 1 | 95° C. | 2 | min |
| 2 | 35 | 98° C. | 10 | sec |
| | | 52° C. | 30 | sec |
| | | 72° C. | 2 | min |
| 3 | 1 | 72° C. | 10 | min |

The PCR reaction relies on two flanking primers (designated “a” and “b”), and one linker-primer (designated “c”). Primer “a” contains 20 nucleotides derived from the 5′ region of the sense strand of the n10 gene (I—SEQ ID NO: 21), whereas primer “b” is similar to the 3′ end of the antisense strand of the Ub gene (II—SEQ ID NO: 20). Importantly, as pfu or Phusion was used as the DNA polymerase, both primers were diluted to a final (standard) concentration of 500 nM. Primer “c” (the linker primer), was used at a concentration of about 2 nM, and its nucleotide sequence reflects the desired junction between the two genes (FIG. 1D). Specifically, the initial 22 nucleotides of primer “c” are identical to the 3′ region of the sense n10 strand, while the remaining 20 nucleotides are derived from the 5′ of the sense strand encoding the Ub gene.

A PCR reaction requires a template and two primers complementary to both ends of the fragment to be amplified. Accordingly, the Ub fragment will be the only fragment amplified at the initial phase of the reaction as it is the only DNA fragment with two complementary primers “b” and “c”. This amplification results in a ubiquitin gene with a 5′ overhang complementary to the 3′ end of the n10 gene. This intermediate product (FIG. 1, fragment B*) may hybridize with the n10 gene (FIG. 1, fragment A). The annealed strands serve both as templates and primers leading to the production of the full-length desired product (III—SEQ ID NO: 22) (FIG. 1, fragment C), which is then amplified by the flanking primers “a” and “b” (FIGS. 1A-B).

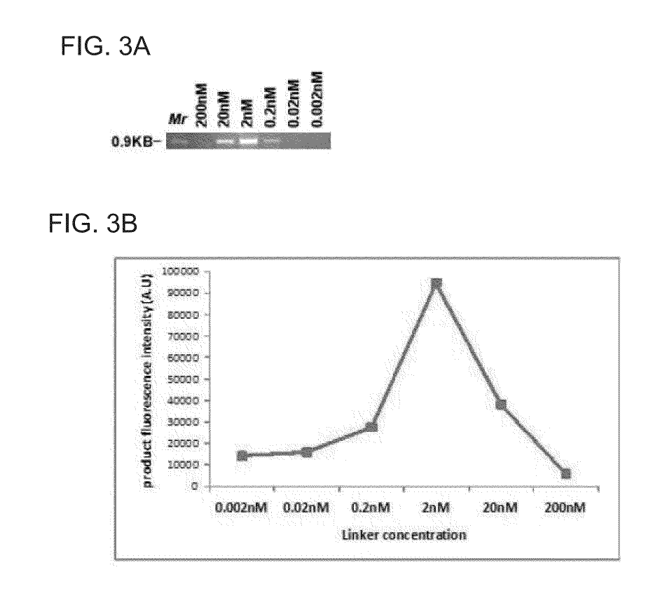

Through extensive calibration assays with various DNA fragments it was found that the linker-induced PCR recombination works best when the linker primer concentration is 2 nM that is about 1:250 of the flanking primers “a” and “b”. A representative assay demonstrating the efficiency of the PCR recombination reaction as a function of the linker primer concentration is given in FIGS. 3A-B. Using the reaction conditions set forth in Tables 4 and 5 and the modified primers (specifically Rpn10-ubiquitin linker with 1T—GGAAAGGTTAAGACAGCAGCAATGGTGGAGGCAAGACTTTGAC—SEQ ID NO: 8; Rpn10-ubiquitin linker with 2T—GGAAAGGTTAAGACAGCAGCAATTGGTGGAGGCAAGACTTTGAC—SEQ ID NO: 9; and Rpn10-ubiquitin linker with 3T—GGAAAGGTTAAGACAGCAGCAATTTGGTGGAGGCAAGACTTTGAC—SEQ ID NO: 10) this method can be utilized to fuse genes while shifting the reading frame or introducing mutations at the junction, depending on the linker primer used, as detailed in FIG. 1C.

TABLE 4

| component | amount | |
|---|---|---|
| Distilled water (dH2O) | 36.9 | μl |
| 10X PFU buffer | 5 | μl |
| 100% DMSO | 1.5 | μl |
| dNTPs (2.5 mM each NTP) | 4 | μl |
| RPN10 PCR 10 ng/μl | 0.2 | μl |
| Ubiquitin PCR 10 ng/μl | 0.2 | μl |
| Rpn10 Forward primer 50 nmol/ml | 0.5 | μl |
| Ubiquitin Reverse primer 50 nmol/ml | 0.5 | μl |
| Rpn10-ubiquitin linker with 1T | 0.2 | μl |
| 500 pmol/ml | | |
| Pfu DNA polymerase (2.5 U/μl) | 1 | μl (2.5U) |
| Total reaction volume | 50 | μl |

TABLE 5

| | Number of | | | |
|---|---|---|---|---|
| segment | cycles | temperature | duration | |
| 1 | 1 | 95° C. | 2 | min |
| 2 | 35 | 98° C. | 10 | sec |
| | | 52° C. | 30 | sec |
| | | 72° C. | 2 | min |
| 3 | 1 | 72° C. | 10 | min |

### Example 2

### Linkage of Three Fragments According to Embodiments of the Present Invention

To test if relying on the same PCR principles it is possible to link more than two fragments in a single PCR reaction, the present inventors set out to fuse three arbitrarily-selected gene fragments of yeast (S. cerevisiae) proteosomal subunits, namely RPN10 (IV) (200 bp; SEQ ID NO: 23), RPT5 (V; SEQ ID NO: 24) (200 bp) and RPT2) (765 bp).

Materials and Methods

A PCR reaction was set up as detailed in Table 6, herein below.

TABLE 6

| component | amount | |
|---|---|---|
| Distilled water (dH2O) | 36.5 | μl |
| 10X PFU buffer | 5 | μl |
| 100% DMSO | 1.5 | μl |
| dNTPs (2.5 mM each NTP) | 4 | μl |
| Rpn10 PCR 10 ng/μl | 0.2 | μl |
| Rpt2 PCR 10 ng/μl | 0.2 | μl |
| Rpt5 PCR 10 ng/μl | 0.2 | μl |
| Rpn10 Forward primer 50 nmol/ml | 0.5 | μl |
| Rpt2 Reverse primer 50 nmol/ml | 0.5 | μl |
| Rtp5-Rpt2 Forward linker primer 500 pmol/ml | 0.2 | μl |
| Rpn10-Rpt5 Reverse linker primer 500 pmol/ml | 0.2 | μl |
| Pfu DNA polymerase (2.5 U/μl) | 1 | μl (2.5U) |
| Total reaction volume | 50 | μl |

The sequences of the reaction primers are provided in Table 7, herein below.

TABLE 7

| | | | SEQ |
|---|---|---|---|
| | | | ID |
| symbol | Name | Sequence | NO |
| a | Rpn10 Forward | ATGGTATTGGAAGCTACAGT | 1 |
| | primer | | |
| d | Rpt2 Reverse | TCACAAGTATAAACCTTCTA | 4 |
| e | Rpn10-Rpt5 | GTGTTATCTACGTTTACCGCAT | 11 |
| | Forward linker | CGGTGGGTTGGATAAACA | |
| f | Rpn10-rpt5 | TGTTTATCCAACCCACCGATG | 5 |
| | Reverse linker | CGGTAAACGTAGATAACAC | |
| g | Rp5-rpt2 | TTTTTGAAGCTGGCAGCACCAT | 6 |
| | Forward linker | CGGTGGCTTAGAATCTCA | |
| h | Rp5-rpt2 | TGAGATTCTAAGCCACCGATGGTG | 7 |
| | Reverse linker | CTGCCAGCTTCAAAAA | |

The PCR cycling parameters are set forth in Table 8, herein below.

TABLE 8

| | Number of | | | |
|---|---|---|---|---|
| segment | cycles | temperature | duration | |
| 1 | 1 | 95° C. | 2 | min |
| 2 | 35 | 98° C. | 10 | sec |
| | | 60° C. | 30 | sec |
| | | 72° C. | 2 | min |
| 3 | 1 | 72° C. | 10 | min |

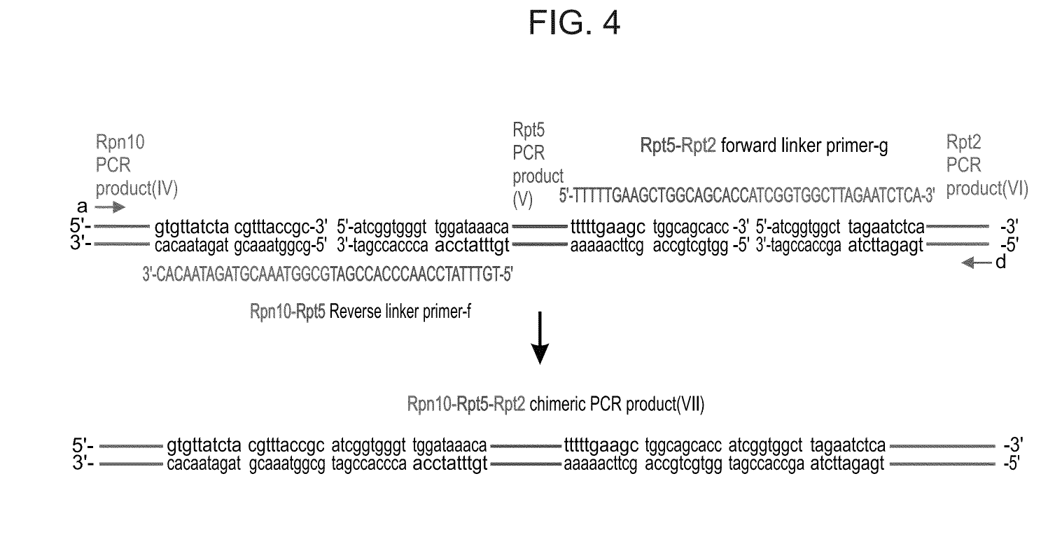

Four primers were used, two flanking primers “a” and “d” and two linker primers “f” and “g” (FIG. 2A). Initially, linker-primers “f” and “g” direct the synthesis of two intermediate products (Fragment A* and fragment C*). As these intermediates are formed, the linker-primers are depleted due to their low initial concentration (1:250). In the next step these products anneal with RPT5 (V—SEQ ID NO: 24) (FIG. 2A, fragment 2) and serve both as templates and primers resulting in generation of two alternative chimeric products (fragments AB*, fragment BC*), which may hybridize with each other or with the first intermediates (FIG. 4). The resulting intermediates converge to form the desired final product Rpn10-Rpt5-Rpt2 (VII—SEQ ID NO: 26), which is amplified via the flanking primers “a” and “d” (FIG. 2A). It is noteworthy that by definition the recombination site of the fragments in the scenarios described above must reside at the end of the templates, otherwise the termini would not be able to serve as primers upon annealing of the intermediate products. To circumvent such a limitation the present inventors use a variation of this method in which primer-linkers suitable for amplification of the desired segments of the three genes are used (FIGS. 5A-B) using the parameters provided in Tables 9 and 10 herein below.

TABLE 9

| component | amount | |
|---|---|---|
| Distilled water (dH2O) | 36.1 | μl |
| 10X PFU buffer | 5 | μl |
| 100% DMSO | 1.5 | μl |
| dNTPs (2.5 mM each NTP) | 4 | μl |
| Rpn10 PCR 10 ng/μl | 0.2 | μl |
| Rpt2 PCR 10 ng/μl | 0.2 | μl |
| Rpt5 PCR 10 ng/μl | 0.2 | μl |
| Rpn10 Forward primer 50 nmol/ml | 0.5 | μl |
| Rpt2 Reverse primer 50 nmol/ml | 0.5 | μl |
| Rpn10-Rpt5 Forward linker primer 500 pmol/ml | 0.2 | μl |
| Rtp5-Rpt2 Forward linker primer 500 pmol/ml | 0.2 | μl |
| Rpn10-Rpt5 Reverse linker primer 500 pmol/ml | 0.2 | μl |
| Rtp5-Rpt2 Reverse linker primer 500 pmol/ml | 0.2 | μl |
| Pfu DNA polymerase (2.5 U/μl) | 1 | μl (2.5U) |
| Total reaction volume | 50 | μl |

TABLE 10

| | Number of | | | |
|---|---|---|---|---|
| segment | cycles | temperature | duration | |
| 1 | 1 | 95° C. | 2 | min |
| 2 | 35 | 98° C. | 10 | sec |
| | | 60° C. | 30 | sec |
| | | 72° C. | 2 | min |
| 3 | 1 | 72° C. | 10 | min |

Once the overhang containing intermediates are formed, amplification of the final product will proceed relying on primers “a” and “d” (FIGS. 6A-B). The efficiency of this reaction is comparable to a similar reaction where the, two step, ‘PCR-driven overlap extension’ method was used to produce the same product by two consecutive PCR reactions.

### Example 3

### Domain Swapping According to Embodiments of the Present Invention

To evaluate if the present method can be applied to the construction of plasmids, the present inventors sought to use it to perform both cloning and domain swapping. An outstanding question in the UPS field is how different ubiquitin-conjugating enzymes (E2's) propagate the assembly of polyubiquitin conjugates through a different lysine residue. For example, the E2-25K conjugates ubiquitin molecules through lysine 48 of ubiquitin, while E2-S promotes ubiquitin polymerization through lysine 11 of ubiquitin. A possible explanation would be that the specific structure of the E2's catalytic site (Active Site Loop, ASL) determines which lysine is conjugated to the adjacent ubiquitin molecule. To examine this possibility, the present inventors set to generate an E2S derivative in which the ASL was swapped with the ASL of E2-25K (FIGS. 7A-B).

Materials and Methods

A PCR reaction was set up as detailed in Table 11, herein below.

TABLE 11

| component | amount | |
|---|---|---|
| Distilled water (dH2O) | 33.9 | μl |
| 5X HF buffer | 10 | μl |
| dNTPs (2.5 mM each NTP) | 4 | μl |
| E2-25K active loop site PCR 10 ng/μl | 0.2 | μl |
| E2-S in pet22 Plasmid 10 ng/μl | 0.2 | μl |
| E2-25K active site loop Forward primer 50 nmol/ml | 0.5 | μl |
| E2-S pet22 Reverse primer 50 nmol/ml | 0.5 | μl |
| 25K-E2S loop linker primer 500 pmol/ml | 0.2 | μl |
| Phusion ® DNA polymerase (2 U/μl) | 0.5 | μl (1U) |
| Total reaction volume | 50 | μl |

Following the PCR reaction, the products were digested for 1 hour by DpnI endonuclease (Fermentas) and subsequently separated on 1% agarose gel. A fragment of the correct size was excised from the gel and purified using a gel clean kit (Promega). The cleaned PCR product was complemented with nucleotide and its termini were Phosphorylated by T4 Poly Nucleotide Kinase (PNK) for 30 minutes. The PNK was then heat inactivated, cooled and used directly for self-ligation (overnight). 10 μl of the ligation mixture were then used to transform E. coli competent cells (Top10). The transformed bacteria were spread on LB plate complemented with 200 μg/ml Amp. Several colonies were inoculated and the plasmids were sequenced.

The sequences of the reaction primers are provided in Table 12, herein below.

TABLE 12

| | | | SEQ ID |
|---|---|---|---|
| symbol | Name | Sequence | NO |
| L | E2-25K active | ATTAGTTCCGTCACAGGGGC | 12 |
| | site loop | | |
| | Forward | | |
| m | E2S-Pet22 | GTTCGGGTGGAAGATCTTGG | 13 |
| | Reverse | | |
| n | 25K-E2S loop | GAAAGATCAATGGGCAGCT | 14 |
| | linker | GAGCTGGGCATCCGACACGTA | |

The PCR cycling parameters are set forth in Table 13, herein below.

TABLE 13

| | Number of | | | |
|---|---|---|---|---|
| segment | cycles | temperature | duration | |
| 1 | 1 | 98° C. | 2 | min |
| 2 | 35 | 98° C. | 10 | sec |
| | | 60° C. | 30 | sec |
| | | 72° C. | 4 | min |
| 3 | 1 | 72° C. | 10 | min |

The pET22 plasmid carrying the E2S gene (IX—SEQ ID NO: 28) and a PCR fragment of E2-25K ASL (VIII—SEQ ID NO: 27) were used as templates. Two flanking primers “m” and “L” and a linker-primer “n” were also included in the reaction mixture (FIG. 7C). Flanking primer “m” is designed to hybridize with the sense strand of pET22-E2S at the 5′ edge of E2S-ASL and promote the generation of the complementary strand. Linker primer “n” associates with the non-sense strand at the 3′ edge of E2S-ASL (outside of the ASL), and allows the generation of the coding strand. Thus, in the initial PCR cycles a linear form of the pET22 E2S plasmid lacking its ASL with a 5′ 20 bp overhang, complementary to the 3′ of the 25K-ASL, is exclusively generated in limited amounts due to the low concentration of linker primer “n”. The intermediate product anneals to the 25K-ASL to product pet22 E2-S with 25K-ASL (X—SEQ ID NO: 29), which is then further amplified utilizing primers “m” and “L” as shown in FIGS. 7A-B. Following treatment with DpnI the PCR products were then ligated by T4 DNA ligase (to ligate both ends of the chimeric product) and transformed into an E. Coli DH5α strain. Selected clones were sequenced to ascertain the production of the appropriate hybrid. It should be noted that both the flanking primers “m” and “L” were phosphorylated prior to the PCR reaction by T4 PNK, as the 5′ phosphates are necessary for the ligation reaction.

### Example 4

### Cloning According to Embodiments of the Present Invention

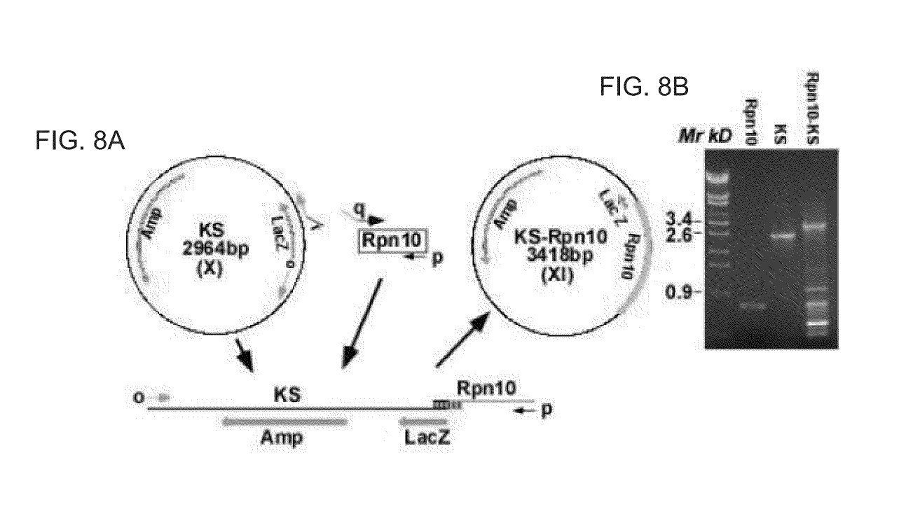

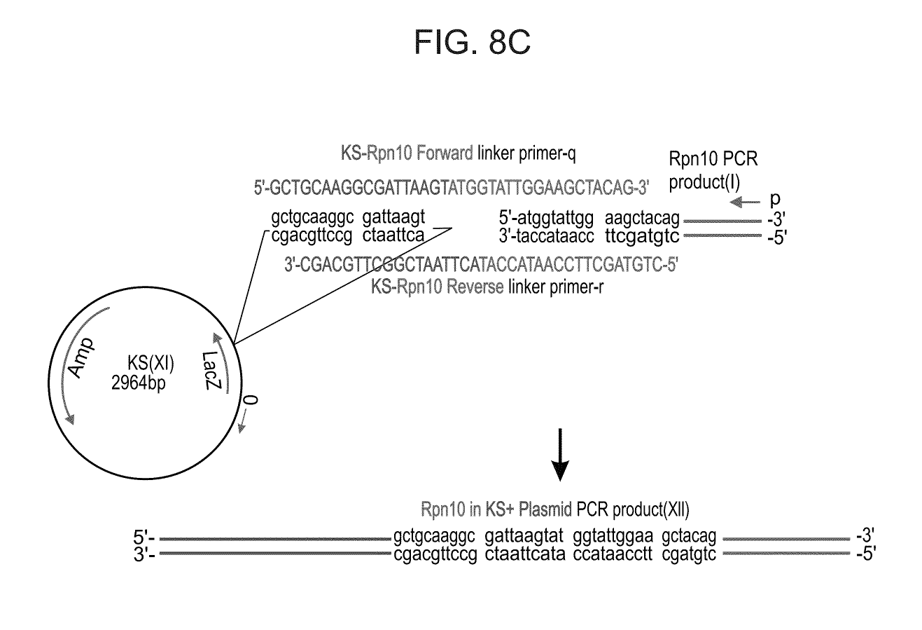

To exemplify that the present method can be utilized for DNA cloning the present inventors aimed to insert the RPN10 gene (I—SEQ ID NO: 21) into the Bluescript-KS (XI—SEQ ID NO: 30) plasmid (FIGS. 8A-B).

Materials and Methods

A PCR reaction was set up as detailed in Table 14, herein below.

TABLE 14

| component | amount | |
|---|---|---|
| Distilled water (dH2O) | 35.7 | μl |
| 10X PFU buffer | 5 | μl |
| 100% DMSO | 1.5 | μl |
| dNTPs (2.5 mM each NTP) | 4 | μl |
| Rpn10 PCR 10 ng/μl | 0.2 | μl |
| Empty KS+ Plasmid 10 ng/ml | 0.2 | μl |
| KS Forward primer 50 nmol/ml | 0.5 | μl |
| Rpn10 Reverse primer 50 nmol/ml | 0.5 | μl |
| KS-rpn10 linker Forward 500 pmol/ml | 0.2 | μl |
| KS-rpn10 linker Reverse 500 pmol/ml | 0.2 | μl |
| Pfu DNA polymerase (2.5 U/μl) | 1 | μl (2.5U) |
| Total reaction volume | 50 | μl |

Following the PCR reaction the products were digested for 1 hour by DpnI endonuclease (Fermentas) and subsequently separated on 1% agarose gel. A fragment of the correct size was excised from the gel and purified using a gel clean kit (Promega). The cleaned PCR product was complemented with nucleotide and its termini were Phosphorylated by T4 Poly Nucleotide Kinase (PNK) for 30 minutes. The PNK was then heat inactivated, cooled and used directly for self ligation (overnight). 10 μl of the ligation mixture were then used to transformed E. coli competent cells (Top10). The transformed bacteria were spread on LB plate complimented with 200 μg/ml Amp, X-gal and IPTG. White colonies were inoculated and the plasmids they carry were sequenced.

The sequences of the reaction primers are provided in Table 15, herein below.

TABLE 15

| | | | SEQ ID |
|---|---|---|---|
| symbol | Name | Sequence | NO |
| o | KS Forward | TACGAGCCGGAAGCATAA | 15 |
| p | Rpn10 Reverse | TTGCTGCTGTCTTAACCTTTC | 16 |
| | primer | | |
| q | KS-rpn10 linker | GCTGCAAGGCGATTAAGT | 17 |
| | Forward | ATGGTATTGGAAGCTACAG | |
| r | KS-rpn10 linker | CTGTAGCTTCCAATACCAT | 18 |
| | Reverse | ACTTAATCGCCTTGCAGC | |

The PCR cycling parameters are set forth in Table 16, herein below.

TABLE 16

| | Number of | | | |
|---|---|---|---|---|
| segment | cycles | temperature | duration | |
| 1 | 1 | 95° C. | 2 | min |
| 2 | 35 | 98° C. | 10 | sec |
| | | 60° C. | 30 | sec |
| | | 72° C. | 4 | min |
| 3 | 1 | 72° C. | 10 | min |

In the experiments presented in Examples 1-3, primer linkers of about 40 bp were used. Given, the annealing temperature of the insertion point in the KS plasmid is predicted to be low and so as to avoid the synthesis of long primers, a combination of two linker primers were used. Accordingly, two overlapping primer-linkers to the same recombination junction were synthesized. The first primer (designated primer “q”) in conjunction with the flanking primer “p” are expected to generate an intermediate of RPN10 with 5′ overhang of 20 bp complementary to the KS recombination site (FIG. 8A). Simultaneously, a second primer linker (designated primer “r”) was employed in conjunction with flanking primer “o” to generate a second intermediate; a KS with 3′ overhang of 20 bp complementary to the 5′ of RPN10 (FIG. 8A). Upon hybridization of these two intermediate products a complementary stretch of 40 nucleotides is generated (in contrast to about 20 bp when a single linker primer is used), thus stabilizing the hybrid. As the intermediate product is being produced, the linker-primers are consumed. The limited amounts of the intermediates formed hybridize and serve as primers and templates for the construction of the full length desired product RPN10 in KS plasmid (XII—SEQ ID NO: 31). The flanking primers “o” and “p” then serve to amplify this linear full length product (FIGS. 8A-C). The PCR product was purified, ligated, and transformed into an E. Coli (DH5α strain) as describe above. Several colonies were sequenced to ensure the correct sequence and orientation.

### Example 4

### Linking of Genomic DNA

In many cases the template used by researchers for cloning originates from a complex mixture, e.g.—genomic DNA or cDNA. Accordingly, the present inventors tested if this technique could be applied directly on genomic DNA.

A PCR reaction was set up as detailed in Table 17, herein below.

TABLE 17

| component | amount | |
|---|---|---|
| Distilled water (dH2O) | 33.9 | μl |
| 5X HF buffer | 10 | μl |
| dNTPs (2.5 mM each NTP) | 4 | μl |
| Rpn10-Rpt5 Forward linker primer 500 pmol/ml | 0.2 | μl |
| Rpn10-Rpt5 Reverse linker primer 500 pmol/ml | 0.2 | μl |
| Rpn10 Forward primer 50 nmol/ml | 0.5 | μl |
| Rpt5 Reverse primer 50 nmol/ml | 0.5 | μl |
| Yeast genome By4741 16.5 ng/μl | 0.2 | μl |
| Phusion ® DNA polymerase (2 U/μl) | 0.5 | μl (1U) |
| Total reaction volume | 50 | μl |
| component | amount | |
| Distilled water (dH2O) | 33.9 | μl |

The sequences of the reaction primers are provided in Table 18, herein below.

TABLE 18

| | | | SEQ ID |
|---|---|---|---|
| symbol | Name | Sequence | NO |
| a | Rpn10 Forward | ATGGTATTGGAAGCTACAGT | 1 |
| | primer | | |
| f | Rpn10-rpt5 | TGTTTATCCAACCCACCGATG | 5 |
| | Reverse linker | CGGTAAACGTAGATAACAC | |
| g | Rp5-rpt2 | TTTTTGAAGCTGGCAGCACCAT | 6 |
| | Forward linker | CGGTGGCTTAGAATCTCA | |
| s | Rpt 5 Reverse | GGTGCTGCCAGCTTCAAAAA | 19 |

The PCR cycling parameters are set forth in Table 19, herein below.

TABLE 19

| | Number of | | | |
|---|---|---|---|---|
| segment | cycles | temperature | duration | |
| 1 | 1 | 98° C. | 2 | min |
| 2 | 70 | 98° C. | 10 | sec |
| | | 60° C. | 30 | sec |
| | | 72° C. | 1 | min |
| 3 | 1 | 72° C. | 10 | min |

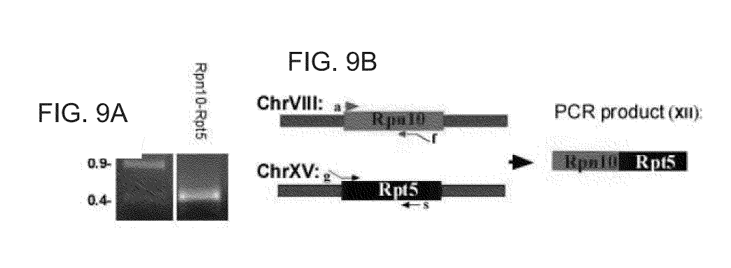

For proof of concept the present inventors selected to fuse two yeast gene products that are located on different chromosomes (RPN10-chr8, RPT5-chr15). As shown in FIGS. 9A-B, using two linker-primers and two flanking primers the present inventors successfully produced a fusion product of the two desired gene fragments (RPN10-RPT5, (XIII—SEQ ID NO: 32)).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of diagnosing a disease in a subject in need thereof, the method comprising:
(a) linking a plurality of non-contiguous DNA fragments from a sample of the subject to generate a polynucleotide product, said linking being effected by contacting said plurality of non-contiguous DNA fragments with a multiplex overlap-extension primer mix under conditions that allow simultaneous linkage of said DNA fragments and amplification of said polynucleotide product, wherein said primer mix comprises two flanking primers and at least one linker primer; and
(b) determining a sequence of said polynucleotide product, wherein said sequence is indicative of the disease.

2. The method of claim 1, wherein at least two of said non-contiguous fragments of DNA comprise a nucleic acid sequence which is indicative of a disease.

3. The method of claim 1, further comprising fragmenting DNA of said sample prior to step (a) so as to generate said plurality of non-contiguous DNA fragments.

4. The method of claim 3, wherein said fragmenting is effected using a restriction enzyme.

5. The method of claim 1, wherein said polynucleotide product is no longer than 1000 base pairs.

6. The method of claim 1, wherein said plurality of non-contiguous fragments comprises two fragments.

7. The method of claim 1, wherein said plurality of non-contiguous fragments comprises three fragments.

8. The method of claim 7, wherein said multiplex overlap-extension primer mix comprises two flanking primers and at least two linker primers.

9. The method of claim 1, wherein a concentration of said at least one linker primer is lower than a concentration of said two flanking primers.

10. The method of claim 1, wherein said determining a sequence is effected using a chain termination method.

11. The method of claim 1, further comprising informing the subject of the results of the diagnosing.

12. The method of claim 1, further comprising performing additional diagnostic tests so as to corroborate the results of the diagnosing.

13. The method of claim 1, wherein said DNA comprises non-transcribed DNA.

14. The method of claim 1, wherein the subject is a fetus.

15. The method of claim 1, wherein the sample comprises amniotic fluid.

16. A method of linking a plurality of non-contiguous fragments of DNA to generate a single polynucleotide product, the method comprising contacting the fragments with a multiplex overlap-extension primer mix, said mix comprising two flanking primers and a number of linker primers being one less than a number of said fragments, under conditions which allow simultaneous linking of said fragments and amplification of said single polynucleotide product, thereby generating the single polynucleotide product.

17. The method of claim 16, wherein said DNA comprises non-transcribed DNA.

18. The method of claim 16, wherein a concentration of said linker primers is lower than a concentration of said flanking primers.

19. A method of linking a plurality of non-contiguous fragments of non-transcribed DNA to generate a single polynucleotide product, the method comprising contacting the fragments with a multiplex overlap-extension primer mix under conditions which allow simultaneous linking of said fragments and amplification of said single polynucleotide product, wherein said primer mix comprises two flanking primers and at least one linker primer, thereby generating the single polynucleotide product.

20. A kit for linking a plurality of non-contiguous fragments of DNA to generate a polynucleotide product, the kit comprising at least two flanking primers and at least one linker primer wherein at least two of said non-contiguous fragments of DNA comprise a nucleic acid sequence which is indicative of a disease.

21. (canceled)
